(19) United States
(12) Patent Application Publication
(10) Pub. No.: US 2006/0205679 A1
(43) Pub. Date: Sep. 14, 2006

(54) Topical and oral formulations of cardiac glycosides for treating skin diseases

(75) Inventors: Robert Streeper, Schertz, TX (US); Chandra U. Singh, San Antonio, TX (US)

Correspondence Address: FULBRIGHT & JAWORSKI L.L.P., 600 CONGRESS AVE., SUITE 2400, AUSTIN, TX, 78701, US

(73) Assignee: Azaya Therapeutics, Inc.

(21) Appl. No.: 11/255,596

(22) Filed: Oct. 21, 2005

Related U.S. Application Data
(60) Provisional application No. 60/621,102, filed on Oct. 22, 2004.

(51) Int. Cl. A61K 31/724; A61K 31/704

(52) U.S. Cl. 514/26; 514/58

(57) ABSTRACT

The present invention provides method, preparation and use of a variety of pharmaceutical compositions containing at least one digitalis glycoside such as oleandrin, odoroside-A, neriifolin, proscillaridin-A, methyl-proscillaridin-A, digitoxin, digoxin alone or at least one digitalis glycoside complexed with cyclodextrins. In another aspect, the present invention provides an effective method to treat diseases in mammals. In yet another aspect, the present invention provides an effective method for treating skin diseases in a human or non-human animal.

### CROSS-REFERENCE TO RELATED APPLICATION

The present invention claims priority to U.S. Provisional Application Ser. No. 60/621,102 filed on Oct. 22, 2004, which is incorporated by reference in its entirety without disclaimer.

### FIELD OF THE INVENTION

The present invention is generally directed to the fields of medicine and pharmacology. The invention is specifically related to pharmaceutical compositions containing oleandrin and other digitalis glycosides for use in the treatment of various diseases. Among the indicated diseases are psoriasis, Crohns disease, lupus, asthma, arthritis, acne, leishmaniasis, neuropathological diseases, decubitus and diabetic ulcers, dandruff, muco-cutaneal disorders due to Tinea spp. infections, Candida spp. infections, Coccidoides spp. infections, moniliasis, dermatological Staphylococcus infections and other diseases such as diabetes, syphilis, neoplasia of the reproductive organs and digestive tract, and meningitis due to fungal and microbial pathogens.

In another aspect, the present invention provides method, preparation and use of a variety of oral and topical formulations containing digitalis glycosides alone, digitalis glycosides complexed with cyclodextrins, or digitialis glycosides alone and or digitalis glycosides complexed with cyclodextrins formulated with other antibacterial, antifungal, or antiviral agents currently having application to the treatment of the aforementioned diseases in humans and other animals. In addition, the specified pharmacologically active agents may be carried in liposomes or other microparticle delivery systems for treating various diseases in humans and other animals.

### BACKGROUND OF THE INVENTION

Cardiac glycosides are found in a broad range of plants and in some animals. Among the plants Nerium oleander, which is the common ornamental oleander plant, and Digitalis purpurea, known as the purple foxglove plant, will be familiar to most readers. Also less widely known plants such as Squill and the colanchoes are known to elaborate cardiac glycosides. Among the animals, certain toads of the genus Bufo produce a class of cardiac glycosides known as the bufadienolides. We now discuss in greater detail the more important biological sources of cardiac glycosides.

Nerium oleander is an evergreen shrub reaching four meters in height. Leaves are 10 to 22 cm long, narrow, untoothed and short-stalked, dark green or grey-green in color. Some cultivars have leaves variegated with white or yellow. All leaves have a prominent mid rib, are leathery in texture and usually arise in groups of three from the stem. The plant produces terminal flower heads, usually pink or white. However, 400 cultivars have been bred and these display a wide variety of different flower color: deep to pale pink, lilac, carmine, purple, salmon, apricot, copper, orange and white (Huxley 1992). Each flower is about 5 cm in diameter and five petalled. The throat of each flower is fringed with long petal like projections. Occasionally double flowers are encountered amongst cultivars. The fruit consists of a long narrow capsule 10 to 12 cm long and 6 to 8 mm in diameter; they open to disperse fluffy seeds. Fruiting is uncommon in cultivated plants. The plant exudes a thick white sap when a twig or branch is broken or cut (Font-Quer 1974, Schvartsman 1979, Lampe & McCann 1985, Pearn 1987). Where the species grows in the wild, such as around the Mediterranean, it occurs along watercourses, in stoney soils and damp ravines. Oleander is widely cultivated in warm temperate and subtropical regions where it grows outdoors in parks, gardens and along roads. Elsewhere, where the plant is grown in colder climates such as in central and western Europe and the western hemisphere, it may be grown as a conservatory or patio plant. N. oleander is cultivated worldwide as an ornamental plant (Kingsbury 1964, Hardin & Arena 1974). In the Mediterranean region, the plant has been used extensively for medicinal purposes. For example, the macerated leaves have been used for itch and hair loss. The fresh leaves have been applied to skin tumors. The decoction of leaves and bark has been used as an antisyphillic. The decoction of leaves has been used as a gargle to strengthen the teeth and gums and as nose drops for children (Dymock 1890, Chopra 1956, Dey 1984 and Kirtikar 1987).

Oleander is one of the digitalis-like plants. The digitalis-like plants produce certain steroidal glycosides with cardiac properties known as digitalis glycosides or cardiac glycosides. Digitalis glycosides are among the most useful groups of drugs in therapeutics (Melero 2000). For example, among the different digitalis glycosides present in Digitalis purpurea, digoxin and its derivatives (acetyl and methyldigoxin) are commonly used in therapeutic preparations for the treatment of cardiovascular ailments.

When ingested, oleandrin is widely distributed in the body. High concentrations of oleandrin have been measured in blood, liver, heart, lung, brain, spleen and kidney in a fatal case of N. oleander extract poisoning (Blum & Rieders 1987). Oleandrin is eliminated one to two weeks from the body (Shaw & Pearn 1979). In 1957, the National Cancer Institute showed that three compounds present in the plant, oleandrin, adynerin and ursolic acid, had significant anti-cancer activity on a number of cultered cancer cell lines. Since then several new chemical compounds have been identified from the methanolic or ethanolic extracts of the plant.

The oleander plant is toxic due to the presence of digitoxin-like steroidal glycosides such as oleandrin. It is estimated that as many as 100 novel chemical substances are present in various parts of the oleander plant (Krasso 1963, Siddiqui 1987-1995, Taylor 1956, Abe 1992, Hanada 1992). Oleandrin C₃₂H₄₈O₉, is the main toxin molucule in the plant. The chemical name of oleandrin is 16β-acetoxy-3β-[(2,6 dideoxy-3-0-methyl-α2-L-arabino-hexopyranosyl)oxy]-14-hydroxy-5β, 14β-card-20(22)-enolide (Reynolds 1989). Oleandrin forms colorless, odorless, acicular crystals which are intensely bitter (Shaw & Pearn 1979). The concentration of oleandrin in the plant tissues is approximately 0.08% by weight (Schvartsman 1979). Oleandrin is almost insoluble in water but is soluble in organic solvents such as ethanol and chloroform. Oleandrin is unstable with respect to light but it is heat stable (Pearn 1987, Reynolds 1989). The chemical structure of oleandrin and the related aglycone oleandrigenin is provided in Formula I. 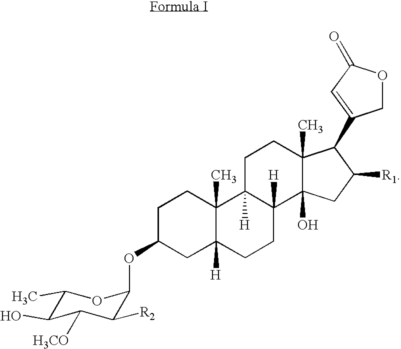
1. Oleandrin: R1=OCOCH3; R2=H
2. Neriifolin: R1=H; R2=OH
3. Odoroside A: R1=H; R2=H
4. Odoroside H: R1=H; R2=OH

The U.S. Pat. No. 5,135,745 describes a procedure for the preparation of the extract of the plant in water. The extraction of oleandrin from Nerium oleander involves boiling the leaves and stems of the plant in water for 2 to 3 hours and filtering off the fibrous plant residues. The chemical constituents of the aqueous extract have been analyzed. It has been found to contain several polysaccharides with molecular weights varying from 2 KD to 30 KD, oleandrin, oleandrogenin as well as a number of other related cardiac glycosides at significantly lower concentrations (Wang 2000). It has been shown that both the water extract of the plant and oleandrin are able to kill human cancer cells, but these compounds are not toxic to murine cancer cells. The cytotoxicity of oleandrin alone was found to be greater than that of the water extract. Canine oral cancer cells treated with water extract of oleander showed intermediate levels of response, with the observation of abnormal metaphases and cell death (apoptosis) resulting from the treatment (Pathak 2000)

Squill, Urginea maritima (L.) Baker, Liliaceae, is a native medicinal and ornamental plant from the Mediterranean area (Kopp 1996, Mitsuhashi 1994, Shoenfeld 1985, Masaru 2001). The bulbs of squill were used in antiquity as a source of rodenticide preparations. Squill was replaced in more recent times by warfarin and other anticoagulant rodenticides. The bulbs of these plants are enormous. After the autumn rains squill send up lush bunches of vigorous leaves. Urginea maritima, and various preparations thereof, have been used to treat neurological pain, skin problems, deep wounds and eye afflictions. Squill also contains active principles that are used in conventional medicine to treat asthma, bronchitis and heart disorders. The plant's name arises from the ability of the root to grow through hard subsoil and reach deeply situated water. It is also traditionaly planted in the vicinity of Arab graves to protect them. The Egyptians call the plant “Ein Sit”, the god who resists the sun, since the plant only blooms in autumn. The Bedouin believe that whenever there is an abundance of Urginea maritima flowers there will be a rainy winter. The plant contains several cardiac glycosides including the bufadienolides proscillaridin A, scillaren A, scillirosid, gammabufotalin, and scillirosidin (Kopp 1990 & 1996, Mitsuhashi 1994, Shoenfeld 1985, Masaru 2001, Majinda 1997, Krenn 1988 & 1994, Krishna Rao 1967, Tanase 1994, Hotta 1994, Verbiscar 1986, Shimada 1979, Jha 1981, Lichti 1973).

The chemical structure of proscillaridin A and its derivative are given in formula II. In the case of proscillaridin A, a pentadienolide lactone ring is at the C17β position instead of a butenolide lactone as in oleandrin. 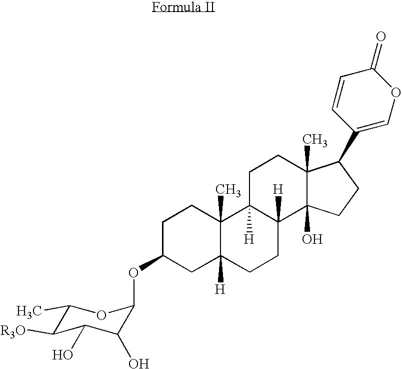
5. Proscillaridin A: R₃═H
6. Methyl-proscillaridin A: R₃═CH₃

Proscillaridin-A is used as a cardiatonic drug in Poland and other countries. Proscillaridin-A is marketed under the brand name Talusin by Knoll Pharma of Switzerland. The oral tablet contains 0.25 mg of proscillardin-A. Proscillaridin A has an oral bioavailability of 20 to 30% in humans.

A list of cardiac glycosides from plants and toads are given in Table 1.

TABLE 1
Fanerogam and Toad species containing digitalis glycosides.

| Species | Cardiotonic glycosides |
|---|---|
| 1. Family Apocynaceae | |
| Nerium oleander | Oleandrin, neriin, neriantin. |
| Nerium odorum | Odoroside A and B. |
| Strophantus gratus, | Ouabain (G-strophantin), |
| S. kombe, | cymarin, sarmentocymarin, |
| S. hispidus, | periplocymarin, K-strophantin. |
| S. sarmentosus, S. emini | |
| Acokanthera schimperi | Ouabain. |
| (A. ouabaïo), | |
| A. venenata, A. abyssinica | |
| Thevetia nereifolia | Thevetin, cerberin, peruvoside. |
| Thevetia yecotli | Thevetosin, thevetin A. |
| Cerbera odollam | Cerberin. |
| Cerbera tanghin | Tanghinin, |
| | deacetyltanghinin, cerberin. |
| Adenium boehmanianum | Echujin, hongheloside G. |
| 2. Family Asclepiadaceae | |
| Periploca graeca | Periplocin. |
| Periploca nigrescens | Strophantidin, strophantidol, |
| | nigrescin. |
| Xysmalobium undulatum | Uzarin. |
| Gomphocarpus fruticosus | Uzarin. |
| Calotropis procera | Calotropin. |
| 3. Family Brassicaceae | |
| Cheiranthus cheiri | Cheiroside A, cheirotoxin. |
| 4. Family Celastraceae | |
| Euonymus europaeus, | Eounoside, euobioside, |
| E. atropur-Pureus | euomonoside. |
| 5. Family Crassulaceae | |
| Kalanchoe lanceolata | Lancetoxin A and B. |
| Kalanchoe tomentosa | Kalanchoside. |
| Kalanchoe tubiflorum | Bryotoxin A-C. |
| Kalanchoe pinnatum | Bryotoxin C, bryophyllin B. |
| Tylecodon wallichii | Cotiledoside. |
| Tylecodon grandiflorus | Tyledoside A-D, F and G. |
| Cotyledon orbiculata | Orbicuside A-C. |
| 6. Family Fabaceae | |
| Coronilla sp. | Alloglaucotoxin, corotoxin, |
| | coroglaucin, glaucorin. |
| 7. Family Iridaceae | |
| Homeria glauca | Scillirosidin derivatives. |
| Moraea polystachya, | Bovogenin A derivatives. |
| M. graminicola | |
| 8. Family Liliaceae | |
| Urginea scilla, | Scillarene A and B, scilliroside, |
| U. maritima | scillarenia, scilliacinoside, |
| | scilliglaucoside, scilliglaucosidin, |
| | scil-liphaeosidin, scilliphaeoside, |
| | scillirosidin, scillirubrosidin, |
| | scillirubroside, proscillaridin A. |
| Urginea rubella | Rubelin. |
| Convalaria majalis | Convalloside, convallatoxin. |
| Bowiea volubilis, | Bovoside A, glucobovoside A, |
| B. kilimand- | bovoruboside. |
| Scharica | |
| 9. Family Moraceae | |
| Antiaria africana, | Antiarin a. |
| A. toxicaria | |
| 10. Family Ranunculaceae | |
| Helleborus niger, H. viridis, | Helleborein, helleborin, hellebrin. |
| H. foeti Dus | |
| Adonis vernalis, | Adonidin, adonin, cymarin, |
| A. aestivalis, A. | adonitoxin. |
| autumnalis, A. flammea | |
| 11. Family Santalaceae | |
| Thesium lineatum | Thesiuside. |
| 12. Family Scrophulariaceae | |
| Digitalis purpurea, D. lanata | Digitoxin, gitoxin, gitalin, digoxin, |
| | F-gitonin, digitonin, lanatoside A-C. |
| 13. Toad Species Genins | |
| Bufo vulgaris | Bufotalin, bufotalinin, bufotalidin. |
| Bufo japonicus | Gamabufagin. |
| Bufo gargarizans | Cinobufagin. |
| Bufo marinus | Marinobufagin. |
| Bufo arenarum | Arenobufagin. |
| Bufo regularis | Regularobufagin. |
| Bufo valliceps | Vallicepobufagin. |
| Bufo quercicus | Quercicobufagin. |
| Bufo viridis | Viridibufagin. |
| Bufo sp. | Pseudobufotalin. |

Cardiac glycosides are used clinically to increase cardiac contractile force in patients with cardiac disorders. Their mechanism of action is well established and involves inhibition of the plasma membrane Na⁺/K⁺-ATPase, leading to alterations in intracellular K⁺ and Ca²⁺ levels.

Na⁺/K⁺-ATPase (EC 3.6.1.37), or the sodium pump, is a carrier enzyme present in almost every animal cell and was discovered by Skou in 1957. Its physiological function is to maintain the Na⁺ and K⁺ electrochemical gradients across the cell membrane, keeping low Na⁺ and high K⁺ intracellular concentrations. Such concentrations of ions, their gradients and the consequent membrane potential drive and modulate a broad range of cellular functions, such as the excitability of nerves and muscle cells, secondary active transport and cellular volume regulation. It is estimated that this enzyme system consumes about 25% of total cellular ATP consumed at rest.

Thus the Na⁺/K⁺-ATPase creates the appropriate Na⁺/K⁺ ratio to maintain the transmembrane potential. The Na⁺ and K⁺ concentrations at rest are: [Na⁺]i=7 to 20 mM, [Na⁺]e=140 mM, [K⁺]i=110 to 120 mM, [K⁺]e=4 to 5 mM. Adenosine triphosphate (ATP) and Mg²⁺ are required for enzyme activity. Binding of the ions to the enzyme, including phosporylation by bound ATP, leads to conformational changes associated to Na⁺ and K⁺ transport. The mechanism of action of cardiac glycosides was put forth by Albers (1967) and Post (1969). The mechanism includes a step in which the enzyme, after expelling 3 Na⁺ and importing in 2 K⁺, becomes susceptible to inhibition by digitalis glycosides or their analogs thus preventing K⁺ binding. Thus inhibiting enzyme activity and further ion transport.

Na⁺/K⁺-ATPase activity is modulated by Na⁺ and K⁺ concentrations, as well as by several steroid hormones, aldosterone, thyroid hormones, catecholamines and peptide hormones such as vasopresin and insulin. Hormone regulation can be carried out at different levels, from the cell membrane to the nucleus, and it can be expressed over short or long time scales (Geering 1997).

Digitalis glycosides are reversible allosteric inhibitors of Na⁺/K⁺-ATPase (Repke 1989). Cardiac glycosides act through inhibition of Na⁺/K⁺-ATPase which subsequently causes the intracellular Ca²⁺ concentration ([Ca²⁺]i) to increase (Thomas 1990). In medical practice digitalis glycosides are administered at doses that produce a moderate degree of enzyme inhibition, roughly 30%, in cardiac muscle. When the muscle cell membrane is depolarized by the action of cardiac glycosides, there are fewer uninhibited Na⁺/K⁺-ATPase enzymes available for the restoration of the Na⁺/K⁺ balance after muscle contraction. The remaining Na⁺/K⁺-ATPase enzymes which are not inhibited by cardiac glycosides will increase their rate of ion transport due to the high [Na⁺]i. For the muscle cell to respond correctly the next triggering nerve impulse, the Na⁺/K⁺ ionic gradient must be restored, although restoration of the gradient will take longer than it would if every Na⁺/K⁺-ATPase were available. This lag causes a temporary increase of [Na⁺]i. This temporary increase of [Na⁺]i causes Ca²⁺ to move into the cell through a Na⁺/Ca²⁺ ion channel. The Na⁺/Ca²⁺ ion channel allows Na⁺ to exit from the cell in exchange for Ca²⁺, or Ca²⁺ exit from the cell in exchange for Na⁺, depending on the prevailing Na⁺ and Ca²⁺ electrochemical gradients (Blaustein 1974). In this way inhibition of the Na⁺/K⁺-ATPase by cardiac glycosides causes the Na⁺/Ca²⁺ exchange to partly reverse resulting in increased intracellular Ca²⁺, which in turn causes increased muscle contractility.

When the concentration of digitalis glycosides reaches toxic levels, enzyme inhibition approaches 60% thus decreasing Na⁺ and K⁺ transport to the extent that the restoration of normal ion transmembrane gradient during diastole is not possible before the next depolarization and contraction cycle. Then, a sustained increase of [Na⁺]i, and thus of [Ca²⁺]i, gives rise to the cardiotoxic effect, i.e. arrhythmia, of these molecules.

Digitalis glycosides represent a very important group of drugs for the treatment of heart failure but have the disadvantage of a narrow therapeutic index, so they must be administered under a strict supervision with continuous monitoring of plasma drug levels. Na⁺/K⁺-ATPase inhibition at therapeutic doses is the mechanism of their positive inotropic effect, since only small changes in [Na⁺]i result in a large change of contractile force (Lee 1985). Apart from their cardiotonic activity, cardiac glycosides also act on other physiological systems, sometimes leading to adverse effects (Gillis 1986).

Cardiac glycosides also have well known antiproliferative effects on tumor cells (Shjratori 1967, Repke 1988 & 1995). The anticancer effects of some cardiac glycosides have been evaluated in short term anticancer animal studies. The conclusion of these experiments is that very high doses, probably in the toxic range, would be needed to obtain clinically significant anticancer effects in humans (Cassady 1980). Recently it has been reported that non-toxic concentrations of digitoxin and digoxin inhibit growth and induce apoptosis in different human malignant cell lines, whereas rapidly proliferating normal cells were not affected, seemingly in contravention of the results of previous investigations (Haux 1999 & 2000). The capability of cardiac glycosides to induce apoptosis has recently been confirmed in other studies (Kawazoe 1999). There is a great difference in toxicity of cardiac glycosides in different species indicating that one can not extrapolate the results from animal models into humans (Repke 1988). In in-vitro experiments the apoptosis inducing effect of digitoxin was found to be greater than that observed for digoxin, and for digitoxin there was a dose response pattern observed, i.e. at higher digitoxin concentrations increased apoptosis was observed. Another recent report on the anticancer effects of cardiac glycosides on tumor cell lines also confirms that digitoxin is more cytotoxic than digoxin (Johansson 2001).

It has been shown that the cardiac glycosides oleandrin, ouabain, and digoxin induce apoptosis in androgen independent human prostate cancer cell lines in-vitro. Cell death was associated with early release of cytochrome-c from mitochondria, followed by proteolytic processing of caspases 8 and 3. Oleandrin also promoted caspase activation, detected by cleavage poly-(ADP-ribose)-polymerase and hydrolysis of the peptide substrate DEVD-pNA. Comparison of the rates of apoptosis in poorly metastatic PC3 M-Pro4 and highly metastatic PC3 M-LN4 cell line subclones demonstrated that cell death was delayed in the latter because of a delay in mitochondrial cytochrome-c release. Single cell imaging of intracellular Ca²⁺ fluxes demonstrated that the pro-apoptotic effects of the cardiac glycosides were linked to their abilities to induce sustained Ca²⁺ increases within the cells. These results show that cardiac glycosides have promise for the treatment of metastatic prostate cancer (McConkey 2000). The saponin digitonin, the aglycone digitoxigenin and five other cardiac glycosides were evaluated for cytotoxicity using primary cultures of tumor cells from patients and a human cell line panel having different cytotoxic drug resistance patterns. Of these seven compounds, proscillaridin-A was the most potent (IC(50): 6.4 to 76 nM), followed by digitoxin, and then ouabain, digoxin, lanatoside C, digitoxigenin and digitonin. Correlation analysis of the log IC(50) values for the cell lines in the panel showed that cytotoxicity was only slightly influenced by resistance mechanisms that involved P-glycoprotein, topoisomerase II, multidrug resistance associated protein pump and glutathione mediated drug resistance. Digitoxin and digoxin expressed selective toxicity against solid tumor cells from patients, while proscillaridin-A expressed no selective toxicity against either solid or hematological tumor cells. The results revealed marked differences in cytotoxicity between the cardiac glycosides, both in potency and selectivity, and exhibit cytotoxicity by mechanisms that differ from those of commonly used anticancer drugs (Johansson 2001).

Further it is known that in-vitro cardiac glycosides inhibit fibroblast growth factor-2 (FGF-2) export through the plasma membrane by interaction with the Na⁺/K⁺-ATPase (Yeh 2001). It has been shown that oleandrin (0.1 ng/mL) produced a 45.7% inhibition of FGF-2 release from PC3 cells and a 49.9% inhibition from DU145 cells. The water extract of the oleander plant (100 ng/mL oleandrin) produced a 51.9 and 30.8% inhibition of FGF-2 release respectively in the two cell lines. These results demonstrate that the water extract, like oleandrin, inhibited FGF-2 export in-vitro through its interaction with Na⁺/K⁺ATPase, from PC3 and DU145 prostate cancer cells in a concentration and time-dependent fashion and may therefore contribute to the antitumor activity of the treatment (Smith 2001).

U.S. Pat. No. 6,071,885 claims cardiac glycosides, specifically digoxin and ouabain, for the treatment of FGF mediated pathophysiological condition in humans. The pathophysiological condition is selected from melanoma, ovarian carcinoma, teratocarcinoma and neuroblastoma. However the patent does not address the Na⁺/K⁺-ATPase pump inhibiting properties of the glycosides which cause the FGF export inhibition (Yeh 2001). For example, Stewart et al. (2000) and Grimes et al. (1995), discuss the importance of the Na⁺/K⁺-ATPase pump inhibiting effects of these glycosides in prostate cancer cell lines. U.S. Pat. No. 6,281,197 similarly claims cardiac glycosides, especially digoxin and ouabain, for the treatment of complications of diabetes involving the inhibition of the export of leaderless FGF proteins. In addition, a literature search using PUBMED (Medline) for cardiac glycoside and diabetes produced more than 300 publications. All of these publications imply the importance of Na⁺/K⁺-ATPase in diabetes mellitus. It has been shown that streptozotocin induced diabetes mellitus in the rat is associated with a substantial increase in ouabain sensitive ATPase activity along most of the nephron (Wald 1986). Further it has been found that there is decrease in Na⁺/K⁺ pump expression in the nerve cells of diabetic rats and that this decrease may be due to atrophy of the axons. In skeletal muscles, myocardium and peripheral nerves the observed decrease in Na⁺/K⁺ pump expression may be important for the pathophysiology of diabetes (Kjeldsen 1987). Diabetic neuropathy is a degenerative complication of diabetes accompanied by an alteration of nerve conduction velocity and Na⁺/K⁺-ATPase activity. Na⁺/K⁺-ATPase activity was significantly lower in sciatic nerve membranes of diabetic rats and significantly restored in diabetic animals that received fish oil supplementation. Diabetes induced a specific decrease the α1 and α3 isoform activity of Na⁺/K⁺-ATPase and protein expression in sciatic nerve membranes (Gerbi 1998). It has been observed that high blood glucose in conjunction with suppressed Na⁺/K⁺ pump activity may induce an increase of Ca²⁺ influx through either Ca²⁺ channels or through reverse Na⁺/Ca²⁺ exchange, possibly leading to the elevation of Ca²⁺ activated voltage dependent K⁺ channels. Both a decrease in inward Na⁺ current and an increase in K⁺ current may result in decreased nerve conduction. In addition, a possible increase of axoplasmic Ca²⁺ concentration may lead to axonal degeneration. These results provide a clue for understanding the pathophysiologic mechanism of diabetic neuropathy (Takigawa 2000).

Further it has been reported that there is a reduction in activity of the ouabain sensitive Na⁺/K⁺-ATPase pump and a coincident reduction in membrane permeability on the diabetic erythrocyte. This effect is most marked in Type 1 diabetics (Jennings 1986). Further it has been found that Na⁺ pumping activity, as estimated from both Na⁺/K⁺-ATPase activity and ouabain binding, was significantly decreased in IDDM and NIDDM subjects, but its insulin sensitivity was retained only in young IDDM subjects (Baldini 1989). It has been observed that VSMC grown in high glucose concentration milieu manifest a decreased Na⁺, K⁺, and Ca²⁺ transport in conjunction with an increase in intracellular concentration of Na⁺ and Ca²⁺. These results suggest that high glucose, per se, may alter membrane permeability to cations, possibly leading to changes in VSMC contractility and/or proliferation. This abnormality due to the diabetic state may be closely linked to the pathogenesis of diabetic angiopathy, thus increasing the risk of hypertension and vascular disease (Kuriyama 1994). Sennoune et al. (2000) studied the effect of streptozotocin induced diabetes on liver Na⁺/K⁺-ATPase in rats. The induced diabetic state resulted in increased Na⁺/K⁺-ATPase activity and an enhanced expression of the beta1 subunit of the Na⁺/K⁺-ATPase. Diabetes mellitus also causes a decrease in membrane fluidity and changes the plasma membrane lipid composition. These results suggest that the increase of Na⁺/K⁺-ATPase activity can be associated with the enhanced expression of the β1 subunit in the diabetic state, but cannot be attributed to changes in membrane fluidity, as typically this enzyme will increase activity in response to an enhancement of membrane fluidity. Further, the level of Na⁺/K⁺-ATPase activity and the number of enzyme units were observed to be about 30% lower in the red blood cells of diabetic patients than in healthy caucasian controls (Raccah 1996).

The ATP binding site, investigated by anisotropy decay studies of the fluorescent probe pyrene isothiocyanate, was modified in women with IDDM and it appears that the Na⁺/K⁺-ATPase of the human placenta is altered in its disposition in IDDM (Zolese 1997). The alteration in small intestinal Na⁺/K⁺-ATPase expression in the chronic diabetic state appears to involve alterations in transcriptional and post-transcriptional processing of the subunits and may likely represent an adaptive response that leads to increased Na⁺ coupled monosaccharide absorption in the context of a perceived state of nutrient depletion (Wild 1999).

U.S. Pat. No. 5,872,103 describes a method for the prevention of mammary tumors by the administration of cardiac glycosides, especially digoxin and digitoxin. The patent is also directed to a method for the prevention of neoplasia using a cardiac glycoside prophylactically to treat an individual who is at risk of developing a neoplastic lesion prior to the development of a clinically observable tumor.

Further, agents that can suppress the activation of nuclear factor-κB (NF-κB) and activator protein-1 (AP-1), may be able to block tumorigenesis and inflammation. Oleandrin blocked tumor necrosis factor (TNF) induced activation of NF-κB in a concentration and time dependent manner. This effect was mediated through inhibition of phosphorylation and degradation of IκBα, an inhibitor of NF-κB. The water extract of Nerium oleander also blocked TNF induced NF-κB activation. Subsequent fractionation and testing of isolated cardiac glycosides present in the extract revealed that this activity was attributable to oleandrin. The effects of oleandrin were not cell type specific because it blocked TNF induced NF-κB activation in a variety of cells. The NF-κB-dependent reporter gene transcription activated by TNF was also suppressed by oleandrin. The TNF induced NF-κB activation cascade involving TNF receptor 1, TNF receptor associated death domain, TNF receptor associated factor 2, NF-κB inducing kinase and IκBα kinase was interrupted at the TNF receptor associated factor 2 and NF-κB inducing kinase levels by oleandrin, thus suppressing NF-κB reporter gene expression. Oleandrin blocked NF-κB activation induced by phorbol esters and bacterial lipopolysaccharide. Oleandrin also blocked AP-1 activation induced by TNF and other stimuli and inhibited the TNF induced activation of c-Jun NH₂-terminal kinase. Overall, the results indicate that oleandrin inhibits activation of NF-κB and AP-1 and their associated kinases. These results may provide a molecular mechanistic basis for the ability of oleandrin to suppress inflammation and perhaps tumorigenesis (Manna 2000).

Rel/NF-κB transcription factors are a family of structurally related eukaryotic transcription factors that are involved in the control of a large number of normal cellular and organismal processes, such as immune and inflammatory responses, developmental processes, cellular growth, and apoptosis. In addition, these factors are active in a number of disease states, including cancer, arthritis, chronic inflammation, asthma, neurodegenerative diseases, and heart disease. Rel/NF-κB transcription factors include a collection of proteins conserved from the fruit fly Drosophila melanogaster to humans. Among the commonly used model organisms, these transcription factors are notably absent in yeast and the nematode Caenorhabditis elegans. In part this may be because one of the primary roles of these factors is to control a variety of physiological aspects of immune and inflammatory responses. A pathway similar to the Rel/NF-κB signaling pathway may also control certain defense responses in plants.

In most cells NF-κB is present as a latent, inactive, IκB bound complex in the cytoplasm. When a cell receives any of a multitude of extracellular signals, NF-κB rapidly dissociates from IκB, enters the nucleus and activates gene expression. Therefore a key step for controlling NF-κB activity is the regulation of the interaction of IκB and NF-κB. Many of the molecular details of this control mechanism are now understood (FIG. 2). Almost all of the signals that lead to the activation of NF-κB converge on a high molecular weight complex that contains a serine specific IκB kinase (IKK). IKK is an unusual kinase in that in most cells IKK contains at least three distinct subunits: IKKα, IKKβ and IKKγ. IKKα and IKKβ are related catalytic kinase subunits, and IKKγ is a regulatory subunit that serves as a conrol mecanism for the catalytic subunits. In the classical or canonical pathway, activation of IKK complex leads to the phosphorylation by IKKβ of two specific serines near the N terminus of IκB, which targets IκB for ubiquitination and degradation by the proteasome. In the non-canonical pathway, the p100-RelB complex is activated by IKKα mediated phosphorylation of the C-terminal region of p100, which leads to degradation of the p100 IkB-like C-terminal sequences to generate p52-RelB. In either pathway, the unmasked Rel/NF-κB complex can then enter the nucleus to activate target gene expression. In the canonical pathway, one of the target genes activated by NF-κB is that which encodes IκB. Newly synthesized IκB can enter the nucleus, remove NF-κB from DNA, and export the complex back to the cytoplasm to restore the original latent state. Thus, the activation of the NF-κB pathway is generally a transient process, lasting from 30 to 60 minutes in most cells.

In some normal cells, such as B cells, some T cells, Sertoli cells and some neurons, NF-κB is constitutively located in the nucleus. In addition, in many cancer cells, including breast cancer, colon cancer, prostate cancer, lymphoid cancers, and probably many others, NF-κB is constitutively active and located in the nucleus. In some cancers, this is due to chronic stimulation of the IKK pathway, while in other cells, such as some Hodgkin's and diffuse large B-cell lymphoma cells, the gene encoding IκB is sometimes mutated and defective. Moreover, several human lymphoid cancer cells have mutations or amplifications of genes encoding Rel/NF-κB transcription factors, which may enable them to accumulate in or rapidly and repeatedly cycle through the nucleus. It is thought that continuous nuclear Rel/NF-κB activity protects cancer cells from apoptosis and in some cases stimulates their growth. Therefore, many current anti-tumor therapies seek to block NF-κB activity as a means for inhibiting tumor growth or sensitizing the tumor cells to more conventional therapies, such as chemotherapy.

TABLE 2
Diseases Associated with NF-κB

| No | Disease | Reference |
|---|---|---|
| 1 | Aging | Chung et al., 2002 |
| 2 | Headaches | Reuter et al., 2003 |
| 3 | Cardiac Hypertrophy | Purcell & Molkentin, 2003 |
| 4 | Muscular Dystrophy (type 2A) | Baghdiguian et al., 1999 |
| 5 | Catabolic disorder | Holmes-McNary, 2002 |
| 6 | Diabetes, Type 1 | Ho & Bray, 1999 |
| 7 | Diabetes, Type 2 | Yuan et al., 2001 |
| 8 | Hypercholesterolemia | Wilson et al., 2000 |
| 9 | Atherosclerosis | Ross et al., 2001 |
| 10 | Heart Disease | Valen et al., 2001 |
| 11 | Ischemia/reperfusion | Toledo-Pereyra & Lopez-Neblina, |
| | | 2002 |
| 12 | Angina Pectoris | Ritchie, 1998 |
| 13 | Pulmonary Disease | Christman et al., 2000 |
| 14 | Acid-induced Lung Inury | Madjdpour et al., 2003 |
| 15 | Renal Disease | Guijarro & Egido, 2001 |
| 16 | Leptospiriosis renal disease | Yang et al., 2001 |
| 17 | Gut Diseases | Neurath et al., 1998 |
| 18 | Skin Diseases | Bell et al., 2003 |
| 19 | Incontinentia pigmenti | Courtois & Israel A, 2000 |
| 20 | Asthma | Pahl & Szelenyi, 2002 |
| 21 | Arthritis | Roshak et al., 2002 |
| 22 | Crohns Disease | Pena & Penate, 2002 |
| 23 | Ocular Allergy | Bielory et al., 2003 |
| 24 | Appendicitis | Pennington et al., 2000 |
| 25 | Pancreatitis | Weber & Adler, 2001 |
| 26 | Periodonitis | Nichols et al., 2001 |
| 27 | Inflammatory Bowel Disease | Dijkstra et al., 2002 |
| 28 | Sepsis | Wratten et al., 2001, |
| | | Abraham, 2003 |
| 29 | Silica-induced | Chen & Shi, 2002 |
| 30 | AIDS (HIV-1) | Hiscott et al., 2001 |
| 31 | Autoimmunity | Hayashi & Faustman, 2000 |
| 32 | Lupus | Kammer & Tsokos, 2002 |
| 33 | Neuropathological Diseases | Cechetto, 2001, |
| | | Mattson & Camandola, 2001 |
| 34 | Sleep apnoea | Lavie, 2003 |
| 35 | Alzheimers Disease | Mattson & Camandola, 2001 |

TABLE 3
Constitutive Activation of NF-κB in Human Cancer Cells

| No | Cancer Type | Reference |
|---|---|---|
| 1 | Breast | Nakshatri et al., 1997; |
| | | Sovak et al., 1997 |
| 2 | Cervix | Nair et al., 2003 |
| 3 | Ovary | Dejardin et al., 1999; |
| | | Huang et al., 2000 |
| 4 | Vulva | Seppanen & Vihko, 2000 |
| 5 | Prostate | Huang et al., 2001b; |
| | | Palayoor et al., 1999 |
| 6 | Kidney | Oya et al., 2001 |
| 7 | Liver | Tai et al., 2000 |
| 8 | Pancreas | Wang et al., 1999; |
| | | Sclabas et al., 2003 |
| 9 | Esophygeal/gastric | Sutter et al., 2004 |
| 10 | Stomach | Sasaki et al., 2001 |
| 11 | Colon | Lind et al., 2001 |
| 12 | Thyroid | Visconti et al., 1997 |
| 13 | Melanoma | Yang & Richmond, 2001 |
| 14 | Head and neck | Ondrey et al., 1999 |
| 15 | Cylindromatosis | Kovalenko et al., 2003; |
| | | Brummelkamp et al., 2003; |
| | | Tropmpouki et al., 2003 |
| 16 | Oral carcinoma | Nakayama et al., 2001 |
| 17 | Astrocytoma/glioblastoma | Hayahsi et al., 2001 |
| 18 | Neuroblastoma | Bian et al., 2002 |
| 19 | Acute lymphoblastic leukemia | Kordes et al., 2000 |
| 20 | Acute myelogenous leukemia | Guzman et al., 2001 |
| 21 | Acute T-cell leukemia | Arima & Tei, 2001 |
| | (HTLV-1) | |
| 22 | Chronic lymphocytic leukemia | Furman et al., 2000 |
| 23 | Burkitts Lymphoma (EBV) | Knecht et al., 2001 |
| 24 | Mantle cell lymphoma | Martinez et al., 2003 |
| 25 | Multiple myeloma | Berenson et al., 2001 |
| 26 | Diffuse large B-cell lymphoma | Davis et al., 2001; |
| | | Shaffer et al., 2002 |
| 27 | Hodgkin's lymphoma | Bargou et al., 1996, 1997; |
| | | Staudt, 2001 |

TABLE 4
In Vitro Transformation of Cells

| No | Cells | Reference |
|---|---|---|
| 1 | BCR-ABL | Reuther et al., 1999 |
| 2 | DBL/DBS | Whitehead et al., 1999 |
| 3 | RAF | Baumann et al., 2000 |
| 4 | RAS | Finco et al., 1997 |
| 5 | RET/PTC3 | Russell et al., 2003 |
| 6 | TEL-JAK2 | Santos et al., 200 |
| 7 | TEL-PDGFR | Besancon et al., 1998 |

There are several diseases in which activation of NF-κB has been implicated. For general reviews on the role of NF-κB in disease, see Aradhya & Nelson (2001), Yamamoto & Gaynor (2002) or Baldwin (2001). In Table 2, the various diseases associated with the activation of NF-κB are provided and in Tables 3 and 4, the implications of NF-κB in cancer are given. As can be seen, numerous diseases are involved in the activation of NF-κB. Thus, compounds that suppress the activation of NF-κB may be potential candidates for treating these diseases.

The water extract of the Nerium oleander plant has been shown to ameliorate the cell proliferative diseases in humans. However, it will be difficult to develop the oleander extract as a parenteral pharmaceutical product suitable for commercialization due to the presence of numerous unknown compounds. Since the anti-tumor activity of the oleander extract has been shown to be due to the presence of oleandrin and oleandrogenin it is desirable to develop oleandrin as an anti-tumor agent. The term cell proliferative diseases is meant here to denote malignant as well as non malignant cell populations which often appear morphologically to differ from the surrounding tissue.

As described above, oleandrin and other cardiac glycosides are extremely toxic due to their cardiotoxic properties and it is believed that the relatively non-toxic nature of the oleander extract is due to the binding of the water insoluble oleandrin and oleandrogenin molecules by the polysaccharides present in the extract. The bound oleandrin and oleandrogenin are soluble in water and the oleandrin is released slowly from the complexing polysaccharides on administration of the extract by intramuscular injection. Also, the amount of oleandrin bound by the extraction procedure is very small, on the order of 2 to 5 micrograms of oleandrin per milligram of oleander extract. Due to their promise as therapies for a wide range of disesases it is desirable to develop alternate delivery vehicles to reduce the toxicity of oleandrin and other digitalis glycosides and thereby increase its therapeutic value. It is highly desirable to develop new procedures capable of increasing the therapeutic value of oleandrin and other digitalis glycosides to treat various diseases in humans.

There are many potential barriers to the effective delivery of a toxic drug in its active form to solid tumors. Most small molecule chemotherapeutic agents have a large volume of distribution on intravenous administration. The result is often a narrow therapeutic index due to a high level of toxicity of these agents to healthy tissues. Through encapsulation of drugs in a macromolecular carrier, such as a liposome, the volume of distribution is significantly reduced and the concentration of drug in the tumor is increased. This strategy results in a decrease in the amount and types of nonspecific toxicities and an increase in the amount of drug that can be effectively delivered to the site of action. Under optimal conditions, the drug is carried within the liposomal internal aqueous space while in the circulation but leaks from the liposome at a sufficient rate to become bioavailable on arrival at the tumor. The liposome protects the drug from metabolism and inactivation in the plasma. Due to size limitations in the transport of large molecules or carriers across healthy endothelium, the drug accumulates to a reduced extent in healthy tissues. However, discontinuities in the endothelium of the tumor vasculature have been shown to result in an increased extravasation of large particulate carriers and, in combination with impaired local lymphatic circulation, results in increased accumulation of liposomal drug at the tumor. All of these factors have contributed to the increased therapeutic index observed with liposomal formulations of some chemotherapeutic agents (Drummond et al. 1999).

Protein microspheres have also been reported in the literature as carriers of pharmacological or diagnostic agents. Microspheres of albumin have been prepared by either heat denaturation or chemical crosslinking for use in drug delivery. Heat denatured microspheres are produced from an emulsified mixture (e.g., albumin, the agent to be incorporated, and a suitable oil) at temperatures between 100° C. and 150° C. The microspheres are then washed with a suitable solvent and stored. Leucuta et al. (1988) describe the method of preparation of heat denatured protein microspheres. The procedure for preparing chemically crosslinked microspheres involves treating the emulsion with glutaraldehyde to crosslink the protein, followed by washing and storage. Lee et al. (1981) and U.S. Pat. No. 4,671,954 teach this method of preparation. The above techniques for the preparation of protein microspheres as carriers of pharmacologically active agents, although suitable for the delivery of water-soluble agents, are incapable of entrapping water insoluble ones. This limitation is inherent in the technique of crosslinking and heat denaturation of the protein component in the aqueous phase of the water in oil emulsion. Any aqueous soluble agent dissolved in the protein containing aqueous phase may be entrapped within the resultant crosslinked or heat denatured protein matrix, but a poorly aqueous soluble or oil soluble agent cannot be incorporated into an aqueous protein matrix formed by these techniques.

U.S. Pat. Nos. 5,439,686 and 5,916,596 teach the methods for the production of particulate vehicles for the intravenous administration of pharmacologically active agents. They disclose methods for the in-vivo delivery of the substantially water insoluble anticancer drug taxol. The suspended particles are encased in a polymeric shell formulated from a biocompatible polymer, and have a diameter of less than about 1 micron. The polymeric shell contains particles of taxol, and optionally a biocompatible dispersing agent in which pharmacologically active agent can be either dissolved or suspended.

Another approach as has been to form a reversible complex between the insoluble drug, such as oleandrin, and a carrier molecule. The characteristics of the carrier molecule are such that the carrier molecule and the reversible complex are soluble in water. Among these known carrier molecules are cyclodextrin compounds. The use of cyclodextrin derivatives as carriers for pharmaceuticals is reviewed by Albers and Muller (Albers 1995).

A variety of improvements in the characteristics of pharmaceutical complexes including various cyclodextrins and cyclodextrin derivatives are disclosed in the following patents, Noda et al., U.S. Pat. No. 4,024,223 methyl salicylate, Szejtli et al. U.S. Pat. No. 4,228,160 indomethacin, Hyashi et al., U.S. Pat. No. 4,232,009 ω-halo-PGI₂ analogs, Matsumoto et al., U.S. Pat. No. 4,351,846 3-hydroxy and 3-oxo prostaglandin analogs, Yamahira et al., U.S. Pat. No. 4,353,793, bencyclane fumarate, Lipari, U.S. Pat. No. 4,383,992 steroids-corticosteroids, androgens, anabolic steroids, estrogens, progestagens complexed with β-cyclodextrin, but not substituted amorphous β cyclodextrins, Nicolau, U.S. Pat. No. 4,407,795 P-hexadecylaminobenzoic acid sodium salt, Tuttle, U.S. Pat. No. 4,424,209 3,4-diisobutyryloxy-N-[3-(4-isobuttyryloxyphenyl)-1-methyl-n-propyl]-β-phenethylamine, Tuttle, U.S. Pat. No. 4,425,336, 3,4-dihydroxy-N-[3-(4-hydroxyphenyl)-1-methyl-n-propyl]-β-phenethylamine, Wagu et al., U.S. Pat. No. 4,438,106 fatty acids EPA and DHA; Masuda et al., U.S. Pat. No. 4,474,881 2-(2-fluoro-4-biphenyl)propionic acid or salt, Shinoda et al., U.S. Pat. No. 4,478,995 acid addition salt of (2′-benzyloxycarbonyl)phenyl trans-4-guanidinomethylcyclo-hexanecaboxylate, Hyashi et al., U.S. Pat. No. 4,479,944 Prostaglandin I₂ analog, Hayashi et al., U.S. Pat. No. 4,479,966, 6,9-methano-prostaglandin I₂ analogs, Harada et al., U.S. Pat. No. 4,497,803 lankacidin-group antibiotic; Masuda U.S. Pat. No. 4,499,085 prostoglandin analog, Szejtli et al., U.S. Pat. No. 4,524,068 piperonyl butoxide, Jones, U.S. Pat. No. 4,555,504 cardiac glycoside, Uekama et al., U.S. Pat. No. 4,565,807 pirprofen, Ueda et al., U.S. Pat. No. 4,575,548 2-nitroxymethyl-6-chloropyridine, Ohwaki et al., U.S. Pat. No. 4,598,070 tripamide anti-hypertensive, Chiesi et al., U.S. Pat. No. 4,603,123 piroxicam (feldene), Hasegawa et al., U.S. Pat. No. 4,608,366 monobenzoxamine, Hiari et al., U.S. Pat. No. 4,659,696 polypeptide, Szejtili et al., U.S. Pat. No. 4,623,641 Prostoglandin I₂ methyl ester, Ninger et al., U.S. Pat. No. 4,663,316. unsaturated phosphorous containing antibiotics including phosphotrienin, Fukazawa et al., U.S. Pat. No. 4,675,395 hinokitol, Shimizu et al., U.S. Pat. No. 4,728,509 3-amino-7-isopropyl-5-oxo-5H-[1]-benzopyrano[2,3-b]pyridine-3-carboxcylic acid, Shibani et al. U.S. Pat. No. 4,728,510 milk component, and Karl et al., U.S. Pat. No. 4,751,095 aspartame.

Among the above-mentioned patents, several indicate that complexes of cyclodextrin with drug substances improve the side effect profile of the drug substance. Szejtli et al., U.S. Pat. No. 4,228,160, disclosed that the frequency and severity of gastric and duodenal erosion and ulceration in rats caused by indomethecin is improved in an oral formulation of a complex of β-cyclodextrin and indomethacin in a 2:1 ratio, but is not improved and in fact worsens in the same oral formulation of a complex of β-cyclodextrin and indomethacin in a 1:1 ratio.

Shimazu et al., U.S. Pat. No. 4,352,793, discloses that a formulation wherein bencyclane fumarate, an anti-convulsive compound with β-cyclodextrin or γ-cyclodextrin yields a complex in which the bencyclane fumarate is an inclusion compound. These complexes, when formulated as a liquid suitable for oral administration were claimed to be less irritating in an isotonic buffered pH 7 solution when administered as drops to the eyes of rabbits, as compared to bencyclane fumarate drops at the same drug concentration in an inactive vehicle. Shimazu et al., also discloses that similar complexes dissolved in rabbit blood in-vitro yielded reduced hemolysis as compared to equal concentrations of bencyclane fumarate alone mixed with rabbit blood.

Masuda et al., U.S. Pat. No. 4,478,811, disclose ophthalmic formulations of β or γ-cyclodextrin complexes of the nonsteroidal anti-inflammatory compound fluorobiphenylacetic acid that are less irritating and painful than the same formulations of fluorobiphenyl acetic acid alone. Uekama et al., U.S. Pat. No. 4,565,807, discloses complexes of α, β and γ cyclodextrin, piprofen and a pharmaceutically acceptable base. Piprofen is an analgesic and anti-inflammatory compound, which is bitter and can cause irritation to the gastrointestinal tract. The complexes disclosed in the patent have improved less bitter taste and are less irritating to the gastrointestinal tract than is the uncomplexed compound piprofen. No preparations suitable for intravenous injection were disclosed.

Lipari, U.S. Pat. No. 4,383,992, discloses topical and ophthalmic solutions comprising a number of different steroid-related compounds including corticosteroids, androgens, anabolic steroids, estrogens, and progestagens complexed with β cyclodextrin. None of the cyclodextrin compounds disclosed by Lipari are substituted or amorphous cyclodextrins. In addition, none or the steroid related compounds disclosed by Lipari are 5β steroids.

Pitha et al., U.S. Pat. No. 4,596,795 discloses complexes containing amorphous hydroxypropyl-β-cyclodextrin and sex hormones, particularly testosterone, progesterone and estradiol as lyophilized powders. These tableted complexes are disclosed as appropriate for administration sublingually or buccally with absorption occurring across the corresponding mucosal membrane. None is administered in solution parenterally. In addition none of the steroid related compounds disclosed by Pitha are 5β steroids.

Pitha et al., U.S. Pat. No. 4,727,064, discloses complexes containing water soluble amorphous substituted cyclodextrin mixtures and drugs with substantially low water solubility which may be lyophilized and the lyophilized powder formed into dosage forms suitable for absorption trans-mucossaly across the oral, buccal or rectal mucosa. The solutions of amorphous, water soluble cyclodextrin alone and not in a complex with a drug substance are disclosed as nonirritating topically, and having low toxicity, both systemic and local, when applied parenterally. These solutions of substituted cyclodextrin alone were tested and shown to be non-lethal when substantial amounts of the cyclodextrin solution were administered intra-peritoneally in mice. A number of categories of drugs are disclosed in Pitha for complex with cyclodextrin derivatives and include inter alia vitamins, salts of retinoic acid, spironolactone, antiviral agents, diuretics, anticoagulants, anticonvulsant and anti-inflammatory agents. Significantly, Pitha, while disclosing that aqueous solutions of 50% cyclodextrin may be used for the purpose of determining solubility of drugs in such solutions does not indicate that such solutions are suitable for intravenous administration. No attempt is made to qualify the solution as to its pyrogenicity. The claimed compositions of matter in the reference contain only cyclodextrin and drug. Liquid or semi-liquid compositions of matter, which are claimed in the reference, appear to be made of cyclodextrins with higher degrees of substitution with hydroxypropyl groups. These cyclodextrins are themselves semi-solid or liquids according to the reference. Thus no aqueous formulations of water, cyclodextrin and drug are disclosed or claimed as suitable for parenteral administration in the reference.

Bekers et al. (1989) describe the investigation of stabilization of mitomycin-C and several related mitomycins by formation of a complex with cyclodextrin. The authors indicate that at the pH ranges studied α and β-cyclodextrin as well as heptakis-(2,6,-di-O-methyl)-β-cyclodextrin and dimethyl-β-cyclodextrin, have no influence on stabilization of mitomycin-C pH dependant degradation. γ-Cyclodextrin is reported as having measurable stabilizing effect on mitomycin in acidic media at pH's above 1.

Bodor, U.S. Pat. No. 5,024,998, and Bodor, U.S. Pat. No. 4,983,586, disclose a series of compositions comprising complexes of hydroxypropyl-β-cyclodextrin (HPCD) complexed to a difficult to solubilize drug, and HPCD complexed to a drug which has first been complexed to a specific class of drug carriers characterized as redox drug carriers. The complex of drug and redox carrier is itself difficult to solubilize and is highly lipophilic due to the presence of pyridine derivatives as part of the redox carrier complex. Bodor U.S. Pat. No. 5,024,998 and U.S. Pat. No. 4,983,586 further claim that a solution of 20 to 50% hydroxypropyl-β-cyclodextrin and lipophilic drug-redox carrier complex, or 20 to 50% hydroxypropyl-β-cyclodextrin and lipophilic and/or water labile drug is useful in a method of decreasing the incidence of precipitation of a lipophilic and/or water labile drug occurring at or near the injection site and/or in the lungs or other organs following parenteral administration. Significantly the Bodor references attribute the precipitation and organ deposition problems associated with parenteral administration of lipophilic drugs to the effects of organic solvents used to solubilize the drug in the parenteral vehicle. The Bodor references additionally state that drugs which are particularly useful in the parenteral composition and methods disclosed therein are those which are relatively insoluble in water but whose water solubility can be substantially improved by formulation with 20 to 50% of the selected cyclodextrin, e.g. HPCD, in water. Significantly no part of Bodor addresses the pyrogenic load on the cyclodextrin or the issue of the pyrogenic effect of the composition when injected parenterally. Thus it is quite clear that the Bodor references are directed to prevention of the phenomenon of precipitation of insoluble drugs and insoluble drug-carrier complexes.

U.S. Pat. No. 5,824,668 discloses the composition of 5β steroid with cyclodextrin suitable for parenteral administration for treating various diseases.

Muller et al. (1992) describes the complex formation of digitoxin with β and γ-cyclodextrins. Uekama et al. (1983) describes the inclusion complexes of the digitalis glycosides digitoxin, digoxin, and methyl digoxin with three cyclodextrins, the α, β, and γ homologues, in water and in the solid state were studied by a solubility method, IR and ¹H-NMR spectroscopy, and X-ray diffractometry. Solid complexes in a molar ratio of 1:4 of the digitalis glycosides with γ-cyclodextrin were prepared and their in-vivo absorption examined. The rapidly dissolving form of the γ-cyclodextrin complex significantly increased plasma levels of digoxin (approximately 5.4-fold) after oral administration to dogs. Ueda et al. (1999) examined the complex formation of digitoxin with δ-cyclodextrin and observed enhanced solubility. Okada and Koizumi (1998) studied the complex formation of digitoxin and digoxin with modified β-cyclodextrins. None of the above studies address the issue of parenteral administration of the digitalis glycosides complexed with cyclodextrins. Further there are no scientific studies on the complex formation of cyclodextrins with oleandrin or other digitalis glycosides such as neriifolin, odoroside and proscillaridin-A.

U.S. Pat. No. 6,407,079 discloses the pharmaceutical compositions comprising inclusion compounds of sparingly water soluble or water labile drugs with β-cyclodextrin ethers or β-cyclodextrin esters and the process for the preparation of such compositions. The patent claims cardiac glycosides as one of the types drugs for the treatment of cardiac disorders. The patent further states that molar ratio of the drug to the cyclodextrin derivative is from about 1:6 to 4:1. The patent claims injectable formulations with 0.45 micron filtering and sterilization. However, the patent does not address the pyrogenicity of the preparation and there is no example of the preparation of the cardiac glycoside-cyclodextrin complex suitable for parenteral administration. According to the patent document, the patent was filed in 1988 and was awarded in 2002. However, the complexation of digitoxin and digoxin with β and γ-cyclodextrins was disclosed to the public by the inventors in 1992 (Muller et al. 1992).

The present invention addresses the topical, parenteral and oral administration of the water soluble formulation of the compound selected from the digitalis glycosides such as oleandrin, odoroside A and H, neriifolin, proscillaridin A, digitoxin, digoxin alone or complexed with cyclodextrins or other suitable carriers administered in conjunction with other currently used pharmacologically active agents which will vary according to the condition being treated, i.e. cardiac glycoside preparations may be co-administered with anti-fungal compounds in cases where the condition being treated has a fungal etiology.

### SUMMARY OF THE INVENTION

The present invention relates to the topical and oral formulations of cardiac glycosides such as oleandrin, digitoxin, digoxin, and proscillaridin-A suitable for oral, parenteral and topical administration. In certain preferred embodiments, the invention relates to the use of the digitalis glycosides to treat skin diseases. The inventors have demonstrated that the formulations of the digitalis glycosides such as oleandrin, digitoxin and proscillaridin-A, disclosed herein, for example, exert healing effects on psoriasis, acne, tinea corpora, tinea capitus, tinea curis, tinea unguum, tinea pedis, dandruff, leishmaniasis, and a variety of fungal and bacterial infections in humans.

An aspect of the present invention relates to a method for the treatment of skin hyperproliferative, inflammatory or infectious disorders in a patient, the method comprising administering to affected skin an effective amount of a composition comprising at least one digitalis glycoside. The digitalis glycoside composition may further comprise a cyclodextrin, preferably an amorphous cyclodextrin. The composition may be comprised in a topical formulation. In certain embodiments, the topical formulation is a cream, lotion, spray, wipe, or drop formulation. The composition may also comprise one or more additional pharmaceutical agents, such as a fungicidal or fungistatic agent, a bacteriocidal or bacteriostatic agent, a viricidal or viristatic agent, a cytotoxic agent, or the like. The additional pharmaceutical agent will generally be included in the composition to complement the digitalis glycoside to treat a specific disease; for example, a fungicidal agent may be included in the composition to treat a skin fungal infection in a subject that occurs alone in the subject or in combination with another skin hyperproliferative, inflammatory or infectious disorder.

In certain embodiments, the formulation will further comprise one or more pharmaceutically acceptable excipients. The excipients may include one or more pharmaceutically acceptable antioxidants. The antioxidant may be ascorbic acid, sodium ascorbate, sodium bisulfite, sodium metabisulfate, curcumin, curcumin derivatives, ursolic acid, resveratrol, resveratrol derivatives, alpha-lipoic acid or monothioglycerol. The excipients may also include one or more pharmaceutically acceptable preservatives and/or buffering agents. The buffering agent may be monobasic and dibasic sodium phosphate, sodium benzoate, potassium benzoate, sodium citrate, sodium acetate or sodium tartrate. The preservative may be methylparaben, methylparaben sodium, propylparaben, propylparaben sodium, benzalkonium chloride or benzthonium chloride. In certain embodiments, the composition comprises one or more pharmaceutically acceptable polysaccharides. The polysaccharide may be dextran sulfate, pectin, modified pectin, insoluble 1,3-β-D glucan, micronized 1,3-β-D glucan, soluble 1,3-β-D glucan, phosphorylated 1,3-β-D glucan, aminated 1,3-β-D glucan and carboxymethylated 1,3-β-D glucan, sulfated 1,3-β-D glucan, insoluble 1,3/1,6-β-D glucan, micronized 1,3/1,6-β-D glucan, soluble 1,3/1,6-β-D glucan, phosphorylated 1,3/1,6-β-D glucan, aminated 1,3/1,6-β-D glucan and carboxymethylated 1,3/1,6-β-D glucan or sulfated 1,3/1,6-β-D glucan. In certain preferred embodiments, the patient is a human.

The digitalis glycoside may be oleandrin, neriifolin, odoroside A and H, ouabain (G-strophantin), cymarin, sarmentocymarin, periplocymarin, K-strophantin, thevetin A, cerberin, peruvoside, thevetosin, thevetin B, tanghinin, deacetyltanghinin, echujin, hongheloside G, honghelin, periplocin, strophantidol, nigrescin, uzarin, calotropin, cheiroside A, cheirotoxin, euonoside, euobioside, euomonoside, lancetoxin A and B, kalanchoside, bryotoxin A-C, bryophyllin B, cotiledoside, tyledoside A-D, F and G, orbicuside A-C, alloglaucotoxin, corotoxin, coroglaucin, glaucorin, scillarene A and B, scilliroside, scilliacinoside, scilliglaucoside, scilliglaucosidin, scillirosidin, scillirubrosidin, scillirubroside, proscillaridin A, rubelin, convalloside, convallatoxin, bovoside A, glucobovoside A, bovoruboside, antiarin A, helleborin, hellebrin, adonidin, adonin, adonitoxin, thesiuside, digitoxin, gitoxin, gitalin, digoxin, F-gitonin, digitonin, lanatoside A-C, bufotalin, bufotalinin, bufotalidin, pseudobufotalin, acetyl-digitoxin, acetyl-oleandrin, beta-methyldigoxin or alpha-methyldigoxin. The digitalis glycoside may be oleandrin, odoroside A or odoroside H, digitoxin, proscillaridin A, methyl-proscillaridin A, or neriifolin.

In certain embodiments, wherein the composition comprises from 0.01 mg per gram to 10 mg per gram by weight of the digitalis glycoside, and the composition preferably comprises from 0.04 mg per gram to 2 mg per gram by weight of the digitalis glycoside. The composition may be administered orally, nasally, topically, rectally or vaginally. In certain embodiments, the amorphous cyclodextrin has a degree of substitution of 2 to 7. In certain embodiments, the ratio by weight of digitalis glycoside to amorphous cyclodextrin is 0.01 to 1.

In certain embodiments, the skin disorder is a hyperproliferative disorder, an inflammatory disorder, or an infectious disorder. The inflammatory disorder may be acne, psoriasis, dandruff, decubitus and diabetic ulcers, skin lesions of lupus erythematosus, erythema multiforme, folliculitis, and rosacea. The infectious disorder may be cutaneous leishmaniasis, Tinea spp. infections, Candida spp. infections, Coccidioides spp. infections, moniliasis, dermatological Staphylococcus infections, infections of the eye and conjunctiva, Treponema infections including syphillus and yaws, dermatological lesions due to Herpes Simplex virus types I and II, and dermatologic pathologies due to tuberculosis infections.

Another aspect of the present invention relates to a method of for the systemic treatment of diabetes types I and II, muscular dystrophy, meningitis due to bacterial or fungal pathogens, pulmonary infections, asthma, leptospirosis renal disease, gut diseases, periodontal diseases, lupus erythematosis, systemic leishmaniasis, systemic Coccidioides spp. infections, Crohns disease, inflammatory bowel disease, irritable bowel syndrome, or human immunodeficiency virus infections (AIDS), the method comprising administering to an affected individual an effective amount of a composition comprising at least one digitalis glycoside. The digitalis glycoside composition may further comprise a cyclodextrin. The cyclodextrin may be an amorphous cyclodextrin. The composition may be comprised in an oral formulation and the formulation is administered orally.

### DETAILED DESCRIPTION OF THE INVENTION

### DEFINITIONS

It is understood that the “digitalis activity” of a molecule refers to the ability of the molecule to inhibit Na⁺/K⁺-ATPase through acting on the digitalis receptor, along with the ability to induce a positive inotropic effect. Such activity is observed in several natural, semisynthetic and synthetic compounds (Thomas 1992). Among the natural compounds, there are three groups; steroidal butenolides and pentadienolides, known as “cardiotonic steroids” or “digitalic compounds” and Erythrophleum alkaloids. The word “digitalis” is often used as a generic word for all cardiotonic steroids. Similarly, the receptor for these compounds is generally known as the “digitalis receptor”. Digitalis glycosides are also called cardiac glycosides and are compounds bearing a steroidal genin or aglycone with one or several sugar molecules attached to position C₃. In the case of the toad venoms, the sugar is replaced by a suberylarginine moeity.

As used herein, the term “micron” refers to a unit of measure of one one-thousandth of a millimeter.

As used herein, the term “nm” or the term “nanometer” refers to a unit of measure of one one-billionth of a meter.

As used herein, the term “ng” or the term “nanogram” refers to a unit of measure of one one-billionth of a gram.

As used herein, the term “μg” or the term “microgram” refers to a unit of measure of one one-millionth of a gram.

As used herein, the term “mL” refers to a unit of measure of one one-thousandth of a liter.

As used herein, the term “mM” refers to a unit of measure of one one-thousandth of a mole.

As used herein, the term “biocompatible” describes a substance that does not appreciably alter or affect in any adverse way, the biological system into which it is introduced.

As used herein, the term “substantially water insoluble pharmaceutical agent” means biologically active chemical compounds that are poorly soluble or almost insoluble in water. Examples of such compounds are paclitaxel, oleandrin, cyclosporine, digitoxin and the like.

By “cyclodextrin” is meant α, β, or γ-cyclodextrin. Cyclodextrins are described in detail in Pitha et al., U.S. Pat. No. 4,727,064, that is incorporated herein by reference. Cyclodextrins are cyclic oligomers of glucose. These compounds form inclusion complexes with many molecules that can fit into and be reversibly bound within the lipophilic cavity of the cyclodextrin molecule.

The term “cell-proliferative diseases” is meant here to denote malignant as well as non-malignant cell populations which often appear morphologically and histologically atypical and differ from the surrounding tissue.

By “amorphous cyclodextrin” is meant non-crystalline mixtures of cyclodextrins wherein the mixture is prepared from α, β, or γ-cyclodextrin or any derivatives thereof, both natural and synthetic. In general the amorphous cyclodextrin is prepared by non-selective additions, especially alkylation of the desired cyclodextrin species. Reactions are carried out to yield mixtures containing a plurality of components thereby preventing crystallization of the cyclodextrin. Various alkylated and hydroxyalkyl-cyclodextrins can be made and of course will vary, depending upon the starting species of cyclodextrin and the addition agent used. Among the amorphous cyclodextrins suitable for compositions according to the invention are hydroxypropyl, hydroxyethyl, glucosyl, maltosyl and maltotriosyl derivatives of β-cyclodextrin, carboxyamidomethyl-β-cyclodextrin, carboxymethyl-β-cyclodextrin, hydroxypropyl-β-cyclodextrin and diethylamino-β-cyclodextrin. In the compositions according to the invention hydroxy-β-cyclodextrin is preferred. The substituted γ-cyclodextrins may also be suitable, including but not necessarily limited to hydroxypropyl, hydroxyethyl, glucosyl, maltosyl and maltotriosyl derivatives of γ-cyclodextrin.

The terms “mixture,” “mix,” and “mixing” or any variants of these terms, when used in the claims and/or specification includes, stirring, blending, dispersing, milling, homogenizing, and other similar methods. The mixing of the components or ingredients of the disclosed compositions can form into a solution. In other embodiments, the mixtures may not form a solution. The ingredients/components can also exist as undissolved colloidal suspensions.

The use of the word “a” or “an” when used in conjunction with the term “comprising” in the claims and/or the specification may mean “one,” but it is also consistent with the meaning of “one or more,” “at least one,” and “one or more than one.”

The use of the term “or” in the claims is used to mean “and/or” unless explicitly indicated to refer to alternatives only or the alternatives are mutually exclusive.

Throughout this application, the term “about” is used to indicate that a value includes the inherent variation of error for the device, the method being employed to determine the value, or the variation that exists among the study subjects.

Digitalis Glycosides

It is contemplated that any digitalis glycoside or digitalis glycoside derivative may be used with the present invention. As stated above, many digitalis glycosides have been identified. The digitalis glycosides of the present invention, preferably in a cyclodextrin complex, may be in the form of pharmaceutically acceptable salts, esters, amides or prodrugs or combinations thereof. However, conversion of inactive ester, amide or prodrug forms to an active form must occur prior to or upon reaching the target tissue or cell. Salts, esters, amides and prodrugs of the active agents may be prepared using standard procedures known to those skilled in the art of synthetic organic chemistry and described, for example, by J. March, Advanced Organic Chemistry: Reactions, Mechanisms and Structure, 4th Ed. (New York: Wiley-Interscience, 1992). For example, acid addition salts are prepared from the free base, typically wherein the neutral form of the drug has a neutral NH₂ group, using conventional means involving reaction with a suitable acid.

Generally, the base form of the drug is dissolved in a polar organic solvent such as methanol or ethanol and the acid is added thereto. The resulting salt either precipitates may be brought out of solution by addition of a less polar, miscible solvent. Suitable acids for preparing acid addition salts include both organic acids (e.g., acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluenesulfonic acid, salicylic acid, and the like) as well as inorganic acids (e.g., hydrochloric acid, hydrobromic acid, sulfuric acid, nitric acid, phosphoric acid, and the like). An acid addition salt may be reconverted to the free base by treatment with a suitable base. Conversely, preparation of basic salts of acid moieties which may be present on a drug are prepared in a similar manner using a pharmaceutically acceptable base such as sodium hydroxide, potassium hydroxide, ammonium hydroxide, calcium hydroxide, trimethylamine, or the like.

Preparation of esters involves functionalization of hydroxyl and/or carboxylic acid groups present within the molecular structure of the drug. The esters are typically acyl-substituted derivatives of free alcohol groups (i.e., moieties derived from carboxylic acids of the formula RCOOH where R is alkyl, and preferably is lower alkyl). Esters can be reconverted to the free acids if desired by using conventional hydrogenolysis or hydrolysis procedures. Preparation of amides and prodrugs can be carried out in an analogous manner. Other derivatives and analogs of the active agents may be prepared using standard techniques known to those skilled in the art of synthetic organic chemistry, or may be deduced by reference to the pertinent literature. In addition chiral active agents may be in enantiomerically pure form, or they may be present as a racemic mixture.

Cyclodextrins

Certain preferred embodiments of the present invention involve the use of compositions comprising at least one digitalis glycoside and at least one cyclodextrin or cyclodextrin derivative. As stated above, cyclodextrin or cyclodextrin derivatives may be used as carrier molecules. The present invention contemplates the use of cyclodextrin to complex the digitalis glycoside for administration to a subject to treat a disease. The cyclodextrin of the compositions according to the invention may be α, β or γ cyclodextrin. α-Cyclodextrin contains six or more glucopyranose units; β-cyclodextrin contains seven glucopyranose units, and γ-cyclodextrin contains eight glucopyranose units. The molecules are believed to exist as truncated cones having a core openings of 4.7 to 5.3 Å, 6.0 to 6.5 Å and 7.5 to 8.3 Å for α, β, or γ-cyclodextrin respectively. The composition according to the invention may comprise a mixture of two or more of the α, β, or γ-cyclodextrins. Usually, however, the composition according to the invention will comprise only one of the α, β, or γ-cyclodextrins. The particular α, β, or γ-cyclodextrin to be used with the particular digitalis type of cardiac glycosides such as oleandrin, digitoxin, digoxin to form the compositions according to the invention may be selected based on the known size of the molecule of the digitalis type of cardiac glycosides such as oleandrin, digitoxin, digoxin and the relative size of the cavity of the cyclodextrin compound and its corresponding complexation affinity.

Generally if the molecule of the digitalis type of cardiac glycosides such as oleandrin, digitoxin, digoxin is relatively large, a cyclodextrin having a larger cavity is used to make the composition according to the invention. Furthermore, if the molecule selected from the digitalis type of cardiac glycosides such as oleandrin, digitoxin or digoxin is administered with an excipient it may be desirable to use a cyclodextrin compound having a larger cavity in the composition according to the invention.

The unmodified α, β, or γ cyclodextrins are less preferred in the compositions according to the invention because the unmodified forms tend to crystallize and are relatively less soluble in aqueous solutions. More preferred for the compositions according to the invention are the α, β, and γ-cyclodextrins that are chemically modified or substituted. Chemical substitution at the 2, 3 and 6 hydroxyl groups of the glucopyranose units of the cyclodextrin rings can yield increases in solubility of the cyclodextrin compound.

Most preferred cyclodextrins in the compositions according to the invention are amorphous cyclodextrin compounds. Amorphous cyclodextrins are non-crystalline mixtures of cyclodextrins wherein the mixture is prepared from α, β, or γ-cyclodextrin. In general, the amorphous cyclodextrin is prepared by non-selective alkylation of the desired cyclodextrin species. Suitable alkylation agents for this purpose include but are not limited to propylene oxide, glycidol, iodoacetamide, chloroacetate, and 2-diethylaminoethlychloride. Reactions are carried out to yield mixtures containing a plurality of components thereby preventing crystallization of the cyclodextrin. Various alkylated cyclodextrins can be made and of course will vary, depending upon the starting species of cyclodextrin and the alkylating agent used. Among the amorphous cyclodextrins suitable for compositions according to the invention are hydroxypropyl, hydroxyethyl, glucosyl, maltosyl and maltotriosyl derivatives of β-cyclodextrin, carboxyamidomethyl-β-cyclodextrin, carboxymethyl-β-cyclodextrin, hydroxypropyl-β-cyclodextrin and diethylamino-β-cyclodextrin. In the compositions of the present invention, hydroxypropyl-β-cyclodextrin is preferred although the α or γ analogs may also be suitable. The particular alkylated α, β or γ-cyclodextrin to be used with the particular compound of digitalis glycosides such as oleandrin, digitoxin, digoxin and proscillaridin-A to form the compositions according to the invention will be selected based on the size of the molecule of the compound and the relative size of the cavity of the cyclodextrin compound. As with the unsubstituted cyclodextrins mentioned above, it may be advantageous to use an alkylated cyclodextrin having a larger cavity when the composition according to the invention also includes an excipient. The use of a particular α, β, or γ-cyclodextrin with a particular digitalis type of cardiac glycosides (such as oleandrin, digitoxin, digoxin, proscillaridin-A, and/or other cardiac glycoside) and excipient in the compositions of the present invention may of course be optimized based on the effectiveness in maintaining the cardiac glycoside in solution.

In certain preferred embodiments, an aqueous preparation of preferably substituted amorphous cyclodextrin and one or more digitalis glycosides may be prepared. The relative amounts of digitalis glycosides and cyclodextrin will vary depending upon the relative amount of each of the digitalis glycosides and the effect of the cyclodextrin on the compound. In general, the ratio of the weight of compound of the digitalis glycosides to the weight of cyclodextrin compound will be in a range between about 1:1 and about 1:100. A weight to weight ratio in a range of about 1:5 to about 1:50 and more preferably in a range of about 1:10 to about 1:20 of a digitalis glycoside to cyclodextrin is believed to be most effective for increased availability of the digitalis glycoside. For example, oleandrin or proscillaridin-A in a ratio of between about 1:10 and about 1:50 drug to amorphous cyclodextrin, wt/wt, and a final concentration of the injection solution of about 0.3 mg/mL of oleandrin is expected to significantly reduce the toxicity as compared to free oleandrin or proscillaridin-A due to the complexation with amorphous cyclodextrin.

Amorphous hydroxypropyl-β-cyclodextrin may be purchased or synthesized. Amorphous hydroxypropyl-β-cyclodextrin may be purchased from a number of vendors including Sigma-Aldrich, Inc. (St. Louis, Mo., USA). In addition, other forms of amorphous cyclodextrin having different degrees of substitution or glucose residue numbers are available commercially. A method for the production of hydroxypropyl-β-cyclodextrin is disclosed in Pitha et al., U.S. Pat. No. 4,727,064, incorporated herein by reference.

To produce the formulations according to the invention, a pre-weighed amount of hydroxypropyl-β-cyclodextrin compound may be placed in a suitable sterile container. Sufficient sterile water may be added to the amorphous cyclodextrin until the desired concentration of hydroxypropyl-β-cyclodextrin is in solution. To this solution a pre-weighed amount of the compound selected from the digitalis type of cardiac glycosides such as oleandrin, digitoxin, digoxin or proscillaridin-A may be added with stirring and with additional standing if necessary until it dissolves and is complexed by the cyclodextrin. The solution may then be frozen to −40° C. and lyophilized to produce the active pharmaceutical ingredient, i.e. the digitalis glycoside-cyclodextrin complex, which can then be used further in the pharmaceutical preparation.

Pharmaceutically Acceptable Polysaccharides

Compositions of the present invention, in certain embodiments, may contain one or more pharmaceutically acceptable polysaccharide. Preferred pharmaceutically acceptable polysaccharides include polysaccharides that exhibit immune system stimulating and/or anti-cancer effects.

Certain polysaccharides exhibit immune-stimulatory effects, and these polysaccharides may be used in conjunction with the present invention. In a preferred embodiment, one or more polysaccharides that exhibit immune stimulatory effects can be administered to a subject in combination with one or more cardiac glycosides to treat a disease. It is known that certain anionic polysaccharides (Baba, 1988), such as dextran sulphate and pustulan sulphate stimulate cell-mediated T-cell immune responses without stimulating antibody mediated immune responses that are B-cell dependent. On the other hand, unmodified polysaccharides stimulate only B-cells and certain other polysaccharides are known to stimulate both T-cell and B-cell responses under certain conditions. The polysaccharides present in water extract of the plant Nerium oleander has been shown to contain galacturonic acids similar to pectin. These polysaccharides are claimed to be immune system stimulants. Thus the formulations of the present invention can contain suitable polysaccharides such as pectin, preferably, modified citrus pectin to provide the stimulant effect.

Glucans are another group of polysaccharides which can produce certain immune-stimulating effects, and glucans may be used with the present invention. It has been previously shown (Glycan Stimulation of Macrophages In-vitro, R. Seljelid, G. Bogwald and A. Lundwall, Experimental Cell Research 131 (1981) 121), that certain glucans, particularly such glucans containing 1,3-bound β-D-glucose entities, activate macrophages in vitro producing cytotoxic effects. Thus the formulations of the present inventions can contain suitable 1,3-β-D glucans and their derivatives such as phosphorylated 1,3-β-D glucan, aminated 1,3-β-D glucan, sulfated 1,3-β-D glucan and carboxymethylated 1,3-β-D glucan to provide the desired immune stimulant effect.

Citrus pectin (CP) is a complex polysaccharide that has shown some anti-cancer effects, and, in certain embodiments, CP and/or CP derivatives may be co-administered with a cardiac glycoside. The effect of CP, a complex polysaccharide rich in galactosyl residues, and its pH modified derivative, modified citrus pectin (MCP) on the experimental metastasis of B16 melanoma cells and prostate cancer cells was analyzed as described in the articles (Platt 1992; Inohara 1994; Pienta 1995 and Raloff 1995). U.S. Pat. Nos. 5,834,442 and 5,895,784 claim the oral administration of modified citrus pectin to treat prostate cancer and melanoma. It was found that co-injection of MCP with the B16-F1 cells xenografted intravenously resulted in a marked inhibition of their ability to colonize the lungs of the injected mice. The hydrokysis of CP results in the generation of smaller sized non-branched carbohydrate chains of similar sugar composition of the unmodified CP. MCP appears to be non-toxic both in-vitro and in-vivo and is sold as nutritional supplement by herbalists and natural medicine vendors.

Pharmaceutical Compositions Comprising Digitalis Glycosides

Compositions employing the formulations of digitalis glycosides such as proscillaridin-A, digitoxin and oleandrin will contain a biologically effective amount of digitalis glycosides. As used herein, a “biologically effective amount” of a compound or composition refers to an amount effective to alter, modulate or reduce disease symptoms. For oral administration, a satisfactory result may be obtained employing the compounds in an amount within the range of from about 0.2 μg/kg to about 100 μg/kg, preferably from about 0.4 μg/kg to about 50 μg/kg and more preferably from about 0.5 μg/kg to about 10 μg/kg alone or in combination with one or more additional anti-tumor, anti-fungal, and/or anti-inflammatory compounds in a therapeutically effective amount employed together in the same dosage form or in separate topical, oral, intramuscular or intravenous dosage forms taken at the same time. The amount of active compounds in such therapeutically useful compositions is such that a suitable dosage will be obtained.

The formulation according to the invention may also be included in other dosage forms. Preferably such other dosage forms will include one or more of the digitalis glycosides. Such dosage forms may be in the form of aqueous suspensions, elixirs, or syrups suitable for oral administration, or compounded as a cream or ointment in a pharmaceutically acceptable topical base allowing the digitalis glycoside compounds to be absorbed across the skin. In addition the formulation according to the invention may be compounded in a lozenge or suppository suitable for trans-mucosal absorption.

For the intended topical mode of administration, the pharmaceutical compositions containing cyclodextrin-digitalis glycoside complex may be in the form of solid, semi-solid or liquid dosage forms, such as, for example, creams, washes, sprays, tablets, suppositories, pills, capsules, powders, liquids, suspensions, or the like, preferably in unit dosage form suitable for single administration of a precise dosage or in multi-dose containers with unit metering capability. The cyclodextrin-digitalis glycoside complex can be lyophilized and the lyophilized powder can be used for preparing solid dosage forms. The compositions will include an effective amount of the selected cyclodextrin-digitalis glycoside complex in combination with a pharmaceutically acceptable carrier and, in addition, may include other pharmaceutical agents, adjuvants, diluents, buffers, etc. The compounds may thus be administered orally, in dosage formulations containing conventional non-toxic pharmaceutically acceptable carriers, adjuvants and vehicles. The equivalent amount of active digitalis glycoside compound administered as cyclodextrin-digitalis glycoside complex will, of course, be dependent on the subject being treated, the subject's weight, the manner of administration and the judgment of the prescribing physician.

For solid compositions (e.g., tablet compositions), conventional nontoxic solid carriers include, for example, pharmaceutical grades of mannitol, lactose, starch, magnesium stearate, sodium saccharin, talc, cellulose, glucose, sucrose, magnesium carbonate, and the like. Liquid pharmaceutically administrable compositions can, for example, be prepared by dissolving, dispersing, etc., an active compound as described herein and optional pharmaceutical adjuvants in an excipient, such as, for example, water, saline, aqueous dextrose, glycerol, ethanol, and the like, to thereby form a solution or suspension. If desired, the pharmaceutical composition to be administered may also contain minor amounts of nontoxic auxiliary substances such as wetting or emulsifying agents, pH buffering agents and the like, for example, sodium acetate, sorbitan mono-laurate, triethanolamine acetate, triethanolamine oleate, etc. Actual methods of preparing such dosage forms are known, or will be apparent, to those skilled in this art. For example, see Remington's Pharmaceutical Sciences, referenced above. For oral administration, the composition will generally take the form of a tablet or capsule, or may be an aqueous or nonaqueous solution, suspension or syrup. Tablets and capsules are preferred oral administration forms. Tablets and capsules for oral use will generally include one or more commonly used carriers such as lactose and corn starch. Lubricating agents, such as magnesium stearate, are also typically added. When liquid suspensions are used, the active agent may be combined with emulsifying and suspending agents. If desired, flavoring, coloring and/or sweetening agents may be added as well. Other optional components for incorporation into an oral formulation herein include, but are not limited to, preservatives, suspending agents, thickening agents, and the like.

Oral dosage units preferably contain a digitalis glycoside in a cyclodextrin-digitalis glycoside complex in the range of about 50 μg of the digitalis glycoside to not more than about 1000 μg preferably in the range of about 100 μg to about 400 μg so long as the dose received by the patient is accompanied by minimal or substantially no undesirable side effects. In embodiments using oleandrin, a particularly preferred oral dosage unit contains about 250 μg equivalent oleandrin, more preferably about 125 μg equivalent oleandrin. In the case of proscillaridin-A, the preferred oral dosage may contain about 250 μg equivalent of proscillaridin-A.

In the case of topical cream, lotion, wipe or spray, the formulation can contain in the range of about 10 μg to about 1000 μg of the digitalis glycoside per gram of the finished formulation. The amount of digitalis glycoside in the cream or lotion or spray will vary depening on the type of digitalis glycoside. In the case of proscillaridin-A-cyclodextrin complex, the topical formulation can contain from about 20 μg to about 500 μg equivalent of proscillaridin-A per gram of the formulation and the preferred composition will contain from about 100 μg to about 250 μg equivalent of proscillaridin-A per gram of the formulation. In the case of oleandrin-cyclodextrin complex, the topical formulation can contain from about 20 μg equivalent of oleandrin per gram of the formulation to about 500 μg equivalent per gram and the preferred composition will contain from about 100 μg to about 250 μg equivalent of oleandrin per gram of the formulation.

In the case of digitoxin-cyclodextrin complex, the topical formulation can contain from about 20 μg equivalent of digitoxin per gram of the formulation to about 1000 μg equivalent per gram and the preferred composition will contain from about 250 μg to about 500 μg equivalent of digitoxin per gram of the formulation.

In a preferred embodiment, a moisturizing skin cream containing a digitalis glycoside can be applied daily. To apply, a generous amount can be first placed on the fingertips or an appropriate delivery device such as a sponge, wipe, or specialized applicator or cloth. Next, the cream can be dabbed onto the skin at the area to be treated. Finally, the cream can be rubbed into the skin until absorbed. Use of the skin cream immediately after washing and towel drying the skin is recommended, in certain preferred embodiments, to maximize the moisturizing and healing effect.

The invention is related to improved formulations and methods of using the same when administering such formulations to patients. As mentioned herein above a number of excipients may be appropriate for use in the formulation which comprise the composition according to the present invention. The inclusion of excipients and the optimization of their concentration for their characteristics such as for example ease of handling or carrier agents will be understood by those ordinarily skilled in the art not to depart from the spirit of the invention as described herein and claimed herein below.

Infectious Diseases

Compositions of the present invention, in certain preferred embodiments, may be used to treat infectious diseases. Infectious diseases include viral, bacterial, protozoan, and fungal diseases. Infectious diseases may affect any human or non-human animal. In a preferred embodiment, the infectuous diseases being treated is an infectious disease that affects the skin of a human or non-human animal. For example, the infectious disease may either directly infect the skin of a subject or result in symptoms (e.g., irritation or inflammation) which affect the skin.

Fungal Diseases

In a preferred embodiment, the compositions of the present invention containing a digitalis glycoside may be used to treat fungal infections (also referred to as fungal diseases), and in an even more preferred embodiment the compositions of the present invention may be used to treat fungal infections of the skin. Fungal diseases are caused by fungal and other mycotic pathogens (some of which are described in Human Mycoses, E. S. Beneke, Upjohn Co.: Kalamazoo, Mich., 1979; Opportunistic Mycoses of Man and Other Animals, J. M. B. Smith, CAB International: Wallingford, UK, 1989; and Scrip's Antifungal Report, by PJB Publications Ltd, 1992); fungal diseases range from mycoses involving skin, hair, or mucous membranes, such as, but not limited to, Aspergillosis, Black piedra, Candidiasis (moniliasis), Chromomycosis, Cryptococcosis, Onychomycosis, or Otitis externa (otomycosis), Phaeohyphomycosis, Phycomycosis, Pityriasis versicolor, ringworm, Tinea barbae, Tinea capitis, Tinea corporis, Tinea cruris, Tinea favosa, Tinea imbricata, Tinea manuum, Tinea nigra (palmaris), Tinea pedis, Tinea unguium, Torulopsosis, Trichomycosis axillaris, White piedra, and their synonyms, to severe systemic or opportunistic infections, such as, but not limited to, Actinomycosis, Aspergillosis, Chromomycosis, Coccidioidomycosis, Cryptococcosis, Entomophthoramycosis, Geotrichosis, Histoplasmosis, Mucormycosis, Mycetoma, Nocardiosis, North American Blastomycosis, Paracoccidioidomycosis, Phaeohyphomycosis, Phycomycosis, pneumocystic pneumonia, Pythiosis, Sporotrichosis, and Torulopsosis, and their synonyms, some of which may be fatal.

Known fungal and mycotic pathogens include, but are not limited to, Absidia spp., Actinomadura madurae, Actinomyces spp., Allescheria boydii, Alternaria spp., Anthopsis deltoidea, Apophysomyces elegans, Arnium leoporinum, Aspergillus spp., Aureobasidium pullulans, Basidiobolus ranarum, Bipolaris spp., Blastomyces dermatitidis, Candida spp., Cephalosporium spp., Chaetoconidium spp., Chaetomium spp., Cladosporium spp., Coccidioides immitis, Conidiobolus spp., Corynebacterium tenuis, Cryptococcus spp., Cunninghamella bertholletiae, Curvularia spp., Dactylaria spp., Epidermophyton spp., Epidermophyton floccosum, Exserophilum spp., Exophiala spp., Fonsecaea spp., Fusarium spp., Geotrichum spp., Helminthosporium spp., Histoplasma spp., Lecythophora spp., Madurella spp., Malassezia furfur, Microsporum spp., Mucor spp., Mycocentrospora acerina, Nocardia spp., Paracoccidioides brasiliensis, Penicillium spp., Phaeosclera dematioides, Phaeoannellomyces spp., Phialemonium obovatum, Phialophora spp., Phoma spp., Piedraia hortai, Pneumocystis carinii, Pythium insidiosum, Rhinocladiella aquaspersa, Rhizomucor pusillus, Rhizopus spp., Saksenaea vasiformis, Sarcinomyces phaeomuriformis, Sporothrix schenckii, Syncephalastrum racemosum, Taeniolella boppii, Torulopsosis spp., Trichophyton spp., Trichosporon spp., Ulocladium chartarum, Wangiella dermatitidis, Xylohypha spp., Zygomyetes spp. and their synonyms. Other fungi that have pathogenic potential include, but are not limited to, Thermomucor indicae-seudaticae, Radiomyces spp., and other species of known pathogenic genera. These fungal organisms are ubiquitous in air, soil, food, decaying food, etc. Histoplasmoses, Blastomyces, and Coccidioides, for example, cause lower respiratory infections. Trichophyton rubrum causes difficult to eradicate nail infections. In some of the patients suffering with these diseases, the infection can become systemic causing fungal septicemia, or brain/meningal infection, leading to seizures and even death.

Viral Diseases

Compositions of the present invention may also be used to treat viral infections (also called viral diseases), preferably viral diseases which affect the skin of a human or non-human animal. Viral diseases include, but are not limited to: influenza A, B and C, parainfluenza (including types 1, 2, 3, and 4), paramyxoviruses, Newcastle disease virus, measles, mumps, adenoviruses, adenoassociated viruses, parvoviruses, Epstein-Barr virus, rhinoviruses, coxsackieviruses, echoviruses, reoviruses, rhabdoviruses, lymphocytic choriomeningitis, coronavirus, polioviruses, herpes simplex, human immunodeficiency viruses, cytomegaloviruses, papillomaviruses, virus B, varicella-zoster, poxviruses, rubella, rabies, picornaviruses, rotavirus, Kaposi associated herpes virus, herpes viruses type 1 and 2, hepatitis (including types A, B, and C), and respiratory syncytial virus (including types A and B).

Bacterial Diseases

Compositions of the present invention may also be used to treat bacterial infections (also called bacterial diseases), preferably bacterial infections of the skin. Bacterial diseases include, but are not limited to, infection by the 83 or more distinct serotypes of pneumococci, streptococci such as S. pyogenes, S. agalactiae, S. equi, S. canis, S. bovis, S. equinus, S. anginosus, S. sanguis, S. salivarius, S. mitis, S. mutans, other viridans streptococci, peptostreptococci, other related species of streptococci, enterococci such as Enterococcus faecalis, Enterococcus faecium, Staphylococci, such as Staphylococcus epidermidis, Staphylococcus aureus, particularly in the nasopharynx, Hemophilus influenzae, pseudomonas species such as Pseudomonas aeruginosa, Pseudomonas pseudomallei, Pseudomonas mallei, brucellas such as Brucella melitensis, Brucella suis, Brucella abortus, Bordetella pertussis, Neisseria meningitidis, Neisseria gonorrhoeae, Moraxella catarrhalis, Corynebacterium diphtheriae, Corynebacterium ulcerans, Corynebacterium pseudotuberculosis, Corynebacterium pseudodiphtheriticum, Corynebacterium urealyticum, Corynebacterium hemolyticum, Corynebacterium equi, etc. Listeria monocytogenes, Nocordia asteroides, Bacteroides species, Actinomycetes species, Treponema pallidum, Leptospirosa species and related organisms. The invention may also be useful against gram negative bacteria such as Klebsiella pneumoniae, Escherichia coli, Proteus, Serratia species, Acinetobacter, Yersinia pestis, Francisella tularensis, Enterobacter species, Bacteriodes and Legionella species and the like.

Protozoan Diseases

Compositions of the present invention may also be used to treat Protozoan infections (also called Protozoan diseases), preferably Protozoan infections which affect the skin of a subject. Protozoan or macroscopic diseases include infection by organisms such as Cryptosporidium, Isospora belli, Toxoplasma gondii, Trichomonas vaginalis, Cyclospora species, for example, and for Chlamydia trachomatis and other Chlamydia infections such as Chlamydia psittaci, or Chlamydia pneumoniae, for example.

Inflammatory and Hyperproliferative Skin Disorders

Certain preferred embodiments of the present invention involve the use of a composition comprising a digitalis glycoside to treat inflammatory skin disorders and/or hyperproliferative skin disorders. The present invention may be used to treat these disorders in a human or non-human animal.

Inflammatory skin disorders are defined here as any disorder resulting in inflammation of the skin. Preferred inflammatory skin disorders include acne, psoriasis, dandruff, decubitus and diabetic ulcers, skin lesions of lupus erythematosus, erythema multiforme, folliculitis, and rosacea.

Hyperproliferative skin disorders include cancerous, pre-cancerous, and non-cancerous disorders of the skin that are characterized by an excessive proliferation of any cell and/or cells that comprise the skin of a subject. Certain hyperproliferative skin disorders may lead to secondary infections of the skin (e.g., a cutaneous fungal infection).

Moisturizing Agents

Certain topical formulations of the present invention may contain moisturizing agents. Non-limiting examples of moisturizing agents that can be used with the compositions of the present invention include amino acids, chondroitin sulfate, diglycerin, erythritol, fructose, glucose, glycerin, glycerol polymers, glycol, 1,2,6-hexanetriol, honey, hyaluronic acid, hydrogenated honey, hydrogenated starch hydrolysate, inositol, lactitol, maltitol, maltose, mannitol, natural moisturization factor, PEG-15 butanediol, polyglyceryl sorbitol, salts of pyrollidone carboxylic acid, potassium PCA, propylene glycol, sodium glucuronate, sodium PCA, sorbitol, sucrose, trehalose, urea, and xylitol.

Other examples include acetylated lanolin, acetylated lanolin alcohol, acrylates/C10-30 alkyl acrylate crosspolymer, acrylates copolymer, alanine, algae extract, aloe barbadensis, aloe-barbadensis extract, aloe barbadensis gel, althea officinalis extract, aluminum starch octenylsuccinate, aluminum stearate, apricot (prunus armeniaca) kernel oil, arginine, arginine aspartate, arnica montana extract, ascorbic acid, ascorbyl palmitate, aspartic acid, avocado (persea gratissima) oil, barium sulfate, barrier sphingolipids, butyl alcohol, beeswax, behenyl alcohol, beta-sitosterol, BHT, birch (betula alba) bark extract, borage (borago officinalis) extract, 2-bromo-2-nitropropane-1,3-diol, butcherbroom (ruscus aculeatus) extract, butylene glycol, calendula officinalis extract, calendula officinalis oil, candelilla (euphorbia cerifera) wax, canola oil, caprylic/capric triglyceride, cardamon (elettaria cardamomum) oil, carnauba (copernicia cerifera) wax, carrageenan (chondrus crispus), carrot (daucus carota sativa) oil, castor (ricinus communis) oil, ceramides, ceresin, ceteareth-5, ceteareth-12, ceteareth-20, cetearyl octanoate, ceteth-20, ceteth-24, cetyl acetate, cetyl octanoate, cetyl palmitate, chamomile (anthemis nobilis) oil, cholesterol, cholesterol esters, cholesteryl hydroxystearate, citric acid, clary (salvia sclarea) oil, cocoa (theobroma cacao) butter, coco-caprylate/caprate, coconut (cocos nucifera) oil, collagen, collagen amino acids, corn (zea mays) oil, fatty acids, decyl oleate, dextrin, diazolidinyl urea, dimethicone copolyol, dimethiconol, dioctyl adipate, dioctyl succinate, dipentaerythrityl hexacaprylate/hexacaprate, DMDM hydantoin, DNA, erythritol, ethoxydiglycol, ethyl linoleate, eucalyptus globulus oil, evening primrose (oenothera biennis) oil, fatty acids, tructose, gelatin, geranium maculatum oil, glucosamine, glucose glutamate, glutamic acid, glycereth-26, glycerin, glycerol, glyceryl distearate, glyceryl hydroxystearate, glyceryl laurate, glyceryl linoleate, glyceryl myristate, glyceryl oleate, glyceryl stearate, glyceryl stearate SE, glycine, glycol stearate, glycol stearate SE, glycosaminoglycans, grape (vitis vinifera) seed oil, hazel (corylus americana) nut oil, hazel (corylus avellana) nut oil, hexylene glycol, honey, hyaluronic acid, hybrid safflower (carthamus tinctorius) oil, hydrogenated castor oil, hydrogenated coco-glycerides, hydrogenated coconut oil, hydrogenated lanolin, hydrogenated lecithin, hydrogenated palm glyceride, hydrogenated palm kernel oil, hydrogenated soybean oil, hydrogenated tallow glyceride, hydrogenated vegetable oil, hydrolyzed collagen, hydrolyzed elastin, hydrolyzed glycosaminoglycans, hydrolyzed keratin, hydrolyzed soy protein, hydroxylated lanolin, hydroxyproline, imidazolidinyl urea, iodopropynyl butylcarbamate, isocetyl stearate, isocetyl stearoyl stearate, isodecyl oleate, isopropyl isostearate, isopropyl lanolate, isopropyl myristate, isopropyl palmitate, isopropyl stearate, isostearamide DEA, isostearic acid, isostearyl lactate, isostearyl neopentanoate, jasmine (jasminum officinale) oil, jojoba (buxus chinensis) oil, kelp, kukui (aleurites moluccana) nut oil, lactamide MEA, laneth-16, laneth-10 acetate, lanolin, lanolin acid, lanolin alcohol, lanolin oil, lanolin wax, lavender (lavandula angustifolia) oil, lecithin, lemon (citrus medica limonum) oil, linoleic acid, linolenic acid, macadamia ternifolia nut oil, magnesium stearate, magnesium sulfate, maltitol, matricaria (chamomilla recutita) oil, methyl glucose sesquistearate, methylsilanol PCA, microcrystalline wax, mineral oil, mink oil, mortierella oil, myristyl lactate, myristyl myristate, myristyl propionate, neopentyl glycol dicaprylate/dicaprate, octyldodecanol, octyldodecyl myristate, octyldodecyl stearoyl stearate, octyl hydroxystearate, octyl palmitate, octyl salicylate, octyl stearate, oleic acid, olive (olea europaea) oil, orange (citrus aurantium dulcis) oil, palm (elaeis guineensis) oil, palmitic acid, pantethine, panthenol, panthenyl ethyl ether, paraffin, PCA, peach (prunus persica) kernel oil, peanut (arachis hypogaea) oil, PEG-8 C12-18 ester, PEG-15 cocamine, PEG-150 distearate, PEG-60 glyceryl isostearate, PEG-5 glyceryl stearate, PEG-30 glyceryl stearate, PEG-7 hydrogenated castor oil, PEG-40 hydrogenated castor oil, PEG-60 hydrogenated castor oil, PEG-20 methyl glucose sesquistearate, PEG40 sorbitan peroleate, PEG-5 soy sterol, PEG-10 soy sterol, PEG-2 stearate, PEG-8 stearate, PEG-20 stearate, PEG-32 stearate, PEG40 stearate, PEG-50 stearate, PEG-100 stearate, PEG-150 stearate, pentadecalactone, peppermint (mentha piperita) oil, petrolatum, phospholipids, polyamino sugar condensate, polyglyceryl-3 diisostearate, polyquaternium-24, polysorbate 20, polysorbate 40, polysorbate 60, polysorbate 80, polysorbate 85, potassium myristate, potassium palmitate, potassium sorbate, potassium stearate, propylene glycol, propylene glycol dicaprylate/dicaprate, propylene glycol dioctanoate, propylene glycol dipelargonate, propylene glycol laurate, propylene glycol stearate, propylene glycol stearate SE, PVP, pyridoxine dipalmitate, quaternium-15, quaternium-18 hectorite, quaternium-22, retinol, retinyl palmitate, rice (oryza sativa) bran oil, RNA, rosemary (rosmarinus officinalis) oil, rose oil, safflower (carthamus tinctorius) oil, sage (salvia officinalis) oil, salicylic acid, sandalwood (santalum album) oil, serine, serum protein, sesame (sesamum indicum) oil, shea butter (butyrospermum parkii), silk powder, sodium chondroitin sulfate, sodium DNA, sodium hyaluronate, sodium lactate, sodium palmitate, sodium PCA, sodium polyglutamate, sodium stearate, soluble collagen, sorbic acid, sorbitan laurate, sorbitan oleate, sorbitan palmitate, sorbitan sesquioleate, sorbitan stearate, sorbitol, soybean (glycine soja) oil, sphingolipids, squalane, squalene, stearamide MEA-stearate, stearic acid, stearoxy dimethicone, stearoxytrimethylsilane, stearyl alcohol, stearyl glycyrrhetinate, stearyl heptanoate, stearyl stearate, sunflower (helianthus annuus) seed oil, sweet almond (prunus amygdalus dulcis) oil, synthetic beeswax, tocopherol, tocopheryl acetate, tocopheryl linoleate, tribehenin, tridecyl neopentanoate, tridecyl stearate, triethanolamine, tristearin, urea, vegetable oil, water, waxes, wheat (triticum vulgare) germ oil, and ylang ylang (cananga odorata) oil.

Antioxidants

Certain topical formulations of the present invention may also contain one or more antioxidants. Non-limiting examples of antioxidants that can be used with the compositions of the present invention include acetyl cysteine, ascorbic acid, ascorbic acid polypeptide, ascorbyl dipalmitate, ascorbyl methylsilanol pectinate, ascorbyl palmitate, ascorbyl stearate, BHA, BHT, t-butyl hydroquinone, cysteine, cysteine HCI, diamylhydroquinone, di-t-butylhydroquinone, dicetyl thiodipropionate, dioleyl tocopheryl methylsilanol, disodium ascorbyl sulfate, distearyl thiodipropionate, ditridecyl thiodipropionate, dodecyl gallate, erythorbic acid, esters of ascorbic acid, ethyl ferulate, ferulic acid, gallic acid esters, hydroquinone, isooctyl thioglycolate, kojic acid, magnesium ascorbate, magnesium ascorbyl phosphate, methylsilanol ascorbate, natural botanical anti-oxidants such as green tea or grape seed extracts, nordihydroguaiaretic acid, octyl gallate, phenylthioglycolic acid, potassium ascorbyl tocopheryl phosphate, potassium sulfite, propyl gallate, quinones, rosmarinic acid, sodium ascorbate, sodium bisulfite, sodium erythorbate, sodium metabisulfite, sodium sulfite, superoxide dismutase, sodium thioglycolate, sorbityl furfural, thiodiglycol, thiodiglycolamide, thiodiglycolic acid, thioglycolic acid, thiolactic acid, thiosalicylic acid, tocophereth-5, tocophereth-10, tocophereth-12, tocophereth-18, tocophereth-50, tocopherol, tocophersolan, tocopheryl acetate, tocopheryl linoleate, tocopheryl nicotinate, tocopheryl succinate, and tris(nonylphenyl)phosphite.

Equivalents

Known and unknown equivalents to the specific compounds, extracts, and active components in such compounds and extracts discussed throughout this specification can be used with the compositions and methods of the present invention. The equivalents can be used as substitutes for the specific compounds, extracts, and active components. The equivalents can also be used to add to the methods and compositions of the present invention. By way of example, equivalents to Monostroma, sea fennel Codium Tomentosum, Chlorella, and/or Ulva Lactuca can be used with the methods and compositions disclosed in this specification. Related species and genuses to the specific compounds and extracts can also be used with the methods and compounds of the present invention.

A person of ordinary skill in the art would be able to recognize and identify acceptable known and unknown equivalents to the specific compounds, extracts, and active components in such compounds and extracts without undue experimentation.

### EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

Topical and Oral Formulations of Oleandrin-Cyclodextrin Complex

200 mg of oleandrin was weighed and placed in a sterile test tube. The oleandrin was dissolved in 3 to 5 mL of purified absolute ethanol. 50 ml of 9.6% solution of hydroxypropyl-β-cyclodextrin was prepared in a 150 mL sterile beaker and the solution was heated to 70 to 80° C. while stirring on a hot plate. The ethanolic solution of oleandrin was slowly added to the beaker with stirring. Within 10 to 30 minutes, the oleandrin dissolved, leaving a clear solution with no accumulation of crystals. Thus, 200 mg was effectively solubilized in 50 ml of 9.6% solution of hydroxypropyl-β-cyclodextrin. The solution was sterile-filtered through a 0.22 μm filter. The solution was frozen below −40° C. and lyophilized. The lyophilized cake was powdered and used for the topical cream, lotion and capsule formulations and the lyophilized powder is denoted as oleandrin-cyclodextrin complex.

A. Preparation of Capsules

The capsule composition is compounded from the following ingredients given in Table 5.

TABLE 5
The Composition for Tablet Preparation

| Ingredient | Amount |
|---|---|
| Oleandrin-cyclodextrin complex | 3.125 parts |
| Lactose | 96.875 parts |
| Micronized Beta-(1,3/1,6) Glucan | 200.00 parts |
| (Baker's Yeast) | |
| R-Alpha Lipoic Acid | 100.00 parts |
| Total | 400.00 parts |

PREPARATION: The oleandrin-cyclodextrin complex is intensively milled with ten times its weight of lactose, the milled mixture is admixed with the remaining amount of the lactose, the micronized beta-glucan and the R-alpha lipoic acid. The mixed powder is again milled and the composition is filled into 400 mg capsule in a conventional capsule loading machine. Each capsule contains 0.125 mg of oleandrin and is an oral dosage unit composition with effective therapeutic action.

B. Prepartion of Topical Cream

A skin cream composition containing oleandrin is shown in Table 6. Table 6 lists the ingredients and proportions of 5 compositions containing oleandrin-cyclodextrin complex, designating the compositions I, II, III, IV and V. Composition II shown in Table 6 is the preferred composition. The top portion of Table 6 shows the ingredients and proportions of the base, and the bottom portion of Table 6 shows the constituents and proportions of the additives. All proportions in Table 1 are in units of percent by weight.

As shown in Table 6, the base consists of oils, waters, and water soluble components as an emulsion. Generally, the base may include any emollients, lubricants, emulsifying agents, thickening agents, humectants, preservatives, antifungal agents, anti-bacterial agents, anti-viral agents, fragrances and wetting agents known in the art to be suitable for use in a skin cream base. Also, any mixing methods known in the art to be suitable for mixing an oil and water emulsion for the purposes of forming a moisturizing skin cream may be used to mix the base ingredients.

Table 6 also shows the additives and their proportions. As shown in Table 6, the skin cream consists of lyophilized oleandrin-cyclodextrin complex, micronized 1,3 beta-glucan and sodium salt of R-alpha-lipioic acid.

TABLE 6
Topical Cream Compositions for Oleandrin

| | COMPOSITIONS | | | |
|---|---|---|---|---|
| INGREDIENT NAME | I % | II % | III % | IV % |
| A. BASE INGREDIENTS | | | | |
| Propylene Glycol | 5.00 | 5.00 | 5.00 | 5.00 |
| Methylparaben | 0.30 | 0.30 | 0.30 | 0.30 |
| Stearate, sold under the | 3.00 | 3.00 | 3.00 | 3.00 |
| Trademarks MYRJ 52S and | | | | |
| PEG-8 STEARATE | | | | |
| Panthenol, sold under the | 2.00 | 2.00 | 2.00 | 2.00 |
| Trademark DL PANTHENOL | | | | |
| 50% LIQUID | | | | |
| Stearyl Alcohol, sold under | 3.00 | 3.00 | 3.00 | 3.00 |
| the Trademark STEARYL | | | | |
| ALCOHOL NF | | | | |
| Glyceryl Stearate, sold | 3.00 | 3.00 | 3.00 | 3.00 |
| under the Trademark | | | | |
| WITCONOL-2400 | | | | |
| Lanolin, sold under the | 2.00 | 2.00 | 2.00 | 2.00 |
| Trademark LANOLIN | | | | |
| SUPRA CORONA | | | | |
| Mineral Oil, sold under the | 5.00 | 5.00 | 5.00 | 4.00 |
| Trademark DRAKEOL 7 - | | | | |
| CAR.MIN. OIL, where Car. Min. | | | | |
| Oil is an abbreviation | | | | |
| for Carnation Mineral Oil | | | | |
| Beeswax, sold under | 5.00 | 5.00 | 5.00 | 4.00 |
| the Trademark WHITE | | | | |
| BEESWAX 422 | | | | |
| Petrolatum, sold under | 10.00 | 9.50 | 9.00 | 9.00 |
| the Trademark | | | | |
| PETROLATUM | | | | |
| SNOW WHITE | | | | |
| Dimethicone, sold under | 1.00 | 1.00 | 1.00 | 1.00 |
| the Trademark | | | | |
| RHODOSIL 47V350 | | | | |
| Propylparaben | .02 | .02 | .02 | .02 |
| Fragrance, sold under | 0.20 | 0.20 | 0.20 | 0.20 |
| the Trademark | | | | |
| DRACENA GA | | | | |
| 1091/A | | | | |
| TOTAL BASE | 39.52 | 39.02 | 38.52 | 36.52 |
| B. ADDITIVES | | | | |
| Oleandrin-Cyclodextrin | 0.125 | 0.25 | 0.375 | 0.25 |
| Complex containing 4% | | | | |
| by weight digitoxin | | | | |
| Micronized Beta-Glucan | 1.00 | 1.00 | 1.00 | 2.00 |
| sold by Biopolymer Engineering | | | | |
| Inc Minnesota | | | | |
| Sodium Salt of | 1.00 | 1.00 | 1.00 | 1.00 |
| R-Alpha Lipoic Acid | | | | |
| TOTAL ADDITIVES | 2.125 | 2.25 | 2.375 | 3.25 |
| Deionized Water qs to | 100 | 100 | 100 | 100 |

Cream composition II in accordance with the above samples was applied to severe cases of psoriasis in the following manner:

The cream is to be applied to the affected areas every day for seven days. Complete remission of the ailment is typically observed at the end of five to seven days. Recurrence is prevented by application to pink spots preliminary to lesions. Application three times a day until such spots disappear is normally observed over two days. The above regimen and results have been observed in 12 cases to date with no adverse side effects indicated.

### Example 2

Topical and Oral Formulations of Digitoxin-Cyclodextrin Complex

500 mg of digitoxin was weighed and placed in a sterile test tube. The digitoxin was dissolved in 5 to 10 mL of purified absolute ethanol. 50 ml of 9% solution of hydroxypropyl-β-cyclodextrin was prepared in a 150 mL sterile beaker and the solution was heated to 70 to 80° C. while stirring on a hot plate. The ethanolic solution of digitoxin was slowly added to the beaker with stirring. Within 10 to 30 minutes, the digitoxin dissolved, leaving a clear solution with no accumulation of crystals. Thus, 500 mg was effectively solubilized in 50 ml of 9% solution of hydroxypropyl-β-cyclodextrin. The solution was sterile-filtered through a 0.22 μm filter. The solution was frozen below −40° C. and lyophilized. The lyophilized cake was powdered and used for the topical cream, lotion and capsule formulations and the lyophilized powder is denoted as digitoxin-cyclodextrin complex.

A. Preparation of Capsules

The capsule composition is compounded from the following ingredients given in Table 7.

TABLE 7
The Composition for Capsule Preparation

| Ingredient | Amount |
|---|---|
| Digitoxin-cyclodextrin complex | 2.50 parts |
| Lactose | 97.50 parts |
| Micronized Beta-(1,3/1,6) Glucan | 200.00 parts |
| (Baker's Yeast) | |
| R-Alpha Lipoic Acid | 100.00 parts |
| Total | 400.00 parts |

PREPARATION: The digitoxin-cyclodextrin complex is intensively milled with ten times its weight of lactose, the milled mixture is admixed with the remaining amount of the lactose, the micronized beta-glucan and the R-alpha lipoic acid. The mixed powder is again milled and the composition is filled into 400 mg capsule in a conventional capsule loading machine. Each capsule contains 0.250 mg of digitoxin and is an oral dosage unit composition with effective therapeutic action.

B. Prepartion of Topical Cream

A skin cream composition containing digitoxin is shown in Table 8. Table 8 lists the ingredients and proportions of four compositions containing digitoxin-cyclodextrin complex, designating the compositions I, II, III, IV. Composition II shown in Table 8 is the preferred composition. The top portion of Table 8 shows the ingredients and proportions of the base, and the bottom portion of Table 8 shows the constituents and proportions of the additives. All proportions in Table 8 are in units of percent by weight.

As shown in Table 8, the base consists of oils, waters, and water soluble components. The base is an emulsion of oils, water and water soluble components. Generally, the base may include any emollients, lubricants, emulsifying agents, thickening agents, humectants, preservatives, antifungal agents, fragrances and wetting agents known in the art to be suitable for use in a skin cream base. Also, any mixing methods known in the art to be suitable for mixing an oil and water emulsion for the purposes of forming a moisturizing skin cream may be used to mix the base ingredients.

Table 8 also shows the additives and their proportions. As shown in Table 8, the skin cream consists of lyophilized digitoxin-cyclodextrin complex, micronized 1,3 beta-glucan and sodium salt of R-alpha-lipioic acid.

TABLE 8
Topical Cream Compositions for Digitoxin

| | COMPOSITIONS | | | |
|---|---|---|---|---|
| INGREDIENT NAME | I % | II % | III % | IV % |
| A. BASE INGREDIENTS | | | | |
| Propylene Glycol | 5.00 | 5.00 | 5.00 | 5.00 |
| Methylparaben | 0.30 | 0.30 | 0.30 | 0.30 |
| Stearate, sold under the | 3.00 | 3.00 | 3.00 | 3.00 |
| Trademarks MYRJ 52S and | | | | |
| PEG-8 STEARATE | | | | |
| Panthenol, sold under the | 2.00 | 2.00 | 2.00 | 2.00 |
| Trademark DL PANTHENOL | | | | |
| 50% LIQUID | | | | |
| Stearyl Alcohol, sold under | 3.00 | 3.00 | 3.00 | 3.00 |
| the Trademark STEARYL | | | | |
| ALCOHOL NF | | | | |
| Glyceryl Stearate, sold | 3.00 | 3.00 | 3.00 | 3.00 |
| under the Trademark | | | | |
| WITCONOL-2400 | | | | |
| Lanolin, sold under the | 2.00 | 2.00 | 2.00 | 2.00 |
| Trademark LANOLIN | | | | |
| SUPRA CORONA | | | | |
| Mineral Oil, sold under the | 5.00 | 5.00 | 5.00 | 4.00 |
| Trademark DRAKEOL 7 - | | | | |
| CAR.MIN. OIL, where Car. Min. | | | | |
| Oil is an abbreviation | | | | |
| for Carnation Mineral Oil | | | | |
| Beeswax, sold under | 5.00 | 5.00 | 5.00 | 4.00 |
| the Trademark WHITE | | | | |
| BEESWAX 422 | | | | |
| Petrolatum, sold under | 10.00 | 9.50 | 9.00 | 9.00 |
| the Trademark | | | | |
| PETROLATUM | | | | |
| SNOW WHITE | | | | |
| Dimethicone, sold under | 1.00 | 1.00 | 1.00 | 1.00 |
| the Trademark | | | | |
| RHODOSIL 47V350 | | | | |
| Propylparaben | .02 | .02 | .02 | .02 |
| Fragrance, sold under | 0.20 | 0.20 | 0.20 | 0.20 |
| the Trademark | | | | |
| DRACENA GA | | | | |
| 1091/A | | | | |
| TOTAL BASE | 39.52 | 39.02 | 38.52 | 36.52 |
| B. ADDITIVES | | | | |
| Digitoxin-Cyclodextrin | 0.10 | 0.25 | 0.50 | 0.25 |
| Complex containing 10% | | | | |
| by weight digitoxin | | | | |
| Micronized Beta-Glucan | 1.00 | 1.00 | 1.00 | 2.00 |
| sold by Biopolymer Engineering | | | | |
| Inc Minnesota | | | | |
| Sodium Salt of | 1.00 | 1.00 | 1.00 | 1.00 |
| R-Alpha Lipoic Acid | | | | |
| TOTAL ADDITIVES | 2.10 | 2.25 | 2.50 | 3.25 |
| Deionized Water qs to | 100 | 100 | 100 | 100 |

Cream composition II in accordance with the above samples was applied to severe cases of psoriasis as described above.

### Example 3

Topical and Oral Formulations of Proscillaridin-A-Cyclodextrin Complex

200 mg of proscillaridin-A was weighed and placed in a sterile test tube and was dissolved in 5 to 10 mL of purified absolute ethanol. 50 ml of 9.6% solution of hydroxypropyl-β-cyclodextrin was prepared in a 150 mL sterile beaker and the solution was heated to 70 to 80° C. while stirring on a hot plate. The ethanolic solution of proscillaridin-A was slowly added to the beaker with stirring. Within 10 to 30 minutes, the proscillaridin-A dissolved, leaving a clear solution with no accumulation of crystals. Thus, 200 mg was effectively solubilized in 50 ml of 9.6% solution of hydroxypropyl-β-cyclodextrin. The solution was sterile filtered through a 0.22 μm filter. The solution was frozen below −40° C. and lyophilized. The lyophilized cake was powdered and used for the topical cream, lotion and capsule formulations and the lyophilized powder is denoted as proscillaridin-A-cyclodextrin complex.

A. Preparation of Capsules

The capsule composition is compounded from the following ingredients given in Table 9.

TABLE 9
The Composition for Tablet Preparation

| Ingredient | Amount |
|---|---|
| Proscillaridin-A-cyclodextrin | 6.25 parts |
| complex | |
| Lactose | 93.75 parts |
| Micronized Beta-(1,3/1,6) Glucan | 200.00 parts |
| (Baker's Yeast) | |
| R-Alpha Lipoic Acid | 100.00 parts |
| Total | 400.00 parts |

PREPARATION: The proscillaridin-A-cyclodextrin complex is intensively milled with ten times its weight of lactose, the milled mixture is admixed with the remaining amount of the lactose, the micronized beta-glucan and the R-alpha lipoic acid. The mixed powder is again milled and the composition is filled into 400 mg-capsule in a conventional capsule loading machine. Each capsule contains 0.250 mg of proscillaridin-A and is an oral dosage unit composition with effective therapeutic action.

B. Prepartion of Topical Cream

A skin cream composition containing proscillaridin-A is shown in Table 10. Table 10 lists the ingredients in the compositions containing proscillaridin-A-cyclodextrin complex. The top portion of Table 10 shows the proportions of the base, and the bottom portion of Table 10 shows the constituents and proportions of the additives. All proportions in Table 10 are in units of percent by weight.

As shown in Table 10, the base consists of a commercially available moisturizing skin lotion. Table 10 also shows the additives and their proportions. As shown in Table 10, the skin cream consists of lyophilized proscillaridin-A-cyclodextrin complex, micronized 1,3 beta-glucan and sodium salt of R-alpha-lipioic acid. The base and the additives were mixed thoroughly in a blender to prepare the cream.

TABLE 10
Topical Cream Composition for Proscillaridin-A

| COMPOSITION | |
|---|---|
| INGREDIENT NAME | % |
| A. BASE INGREDIENT | |
| Curel Moisturizing Lotion | 96.0 |
| sold by The Andrew Jergens Company | |
| Cincinnati, Ohio | |
| B. ADDITIVES | |
| Proscillaridin-A-Cyclodextrin | 0.25 |
| Complex containing 4% | |
| by weight Proscillaridin-A | |
| Micronized Beta-Glucan | 1.00 |
| sold by Biopolymer Engineering Inc | |
| Minnesota | |
| Sodium Salt of | 1.00 |
| R-Alpha Lipoic Acid | |
| TOTAL ADDITIVES | 2.25 |
| Deionized Water qs to | 100 |

Likewise, the amount of active ingredient in these illustrative examples may be varied to achieve the dosage unit range set forth above, and the amounts and nature of the inert pharmaceutical carrier ingredients may be varied to meet particular requirements.

While the present invention has been illustrated with the aid of certain specific embodiments thereof, it will be readily apparent to others skilled in the art that the invention is not limited to these particular embodiments, and that various changes and modifications may be made without departing from the spirit of the invention or the scope of the appended claims.

### REFERENCES

1. Müller B W, Waaler T, Helm H and Anderson A: Complex formation of β- and g-cyclodextrins with digitoxin, Acta Pharm. Nord. 1992; 4: 313-317.
2. Albers E and Müller B W: Cyclodextrin derivatives in pharmaceutics, Crit Rev Ther Drug Carrier Syst. 1995; 12:311-37.
3. Uekama K, Fujinaga T, Hirayama F, Otagiri M, Yamasaki M, Seo H, Hashimoto T, Tsuruoka M: Improvement of the oral bioavailability of digitalis glycosides by cyclodextrin complexation, J Pharm Sci 1983; 72: 1338-41.
4. Okada Y, Koizumi K: Some properties and the inclusion behavior of three positional isomers of 6(1), 6n-di-O-alpha-D-glucosyl-cyclomaltoheptaoses (beta-cyclodextrins), Chem Pharm Bull (Tokyo) 1998; 46: 319-23.
5. Ueda H, Wakamiya A, Endo T, Nagase H, Tomono K, Nagai T: Interaction of cyclomaltononaose (delta-CD) with several drugs, Drug Dev Ind Pharm 1999; 25: 951-4.
6. Bekers et al.: “Stabilization of Mitomycins on Complexation With Cyclodextins in Aqueous Acidic Media”, Intl. J. of Pharmaceutics, 52, 239 (1989).
7. Abe F and Yamauchi T: Phytochemistry, 31, 2459 (1992).
8. Allen T M, Hansen C, Martin F J, Redemann C and Yau-Young A: Liposome containing a synthetic lipid derivative of poly(ethylene glycol) show prolonged circulation half-lives in vivo. Biochim. Biophys. Acta, 1066, 29-36 (1991).
9. Baba et al.: Proc. Nat. Acad. Sci., USA, 85, 6132 (1988).
10. Baba et al.: Antiviral Res., 9, 335 (1988).
11. Blum L M and Rieders F: Oleandrin distribution in a fatality from rectal and oral Nerium oleander extract administration. J Anal Toxicol, 1987, 11(5): 219-221.
12. Chopra R N, Nayar S L and Chopra I C: Glossary of Indian Medicinal Plants, CSIR, New Delhi (1956), page 175.
13. Dey K L and Bahadur R: Indigenous Drugs of India, International Book Publishers, India (1984).
14. Dobereiner H G, Kas J, Noppl D, Sprenger I & Sackmann E: Biophys. J. 65, 1396-140 (1993).
15. Drummond D C et al: Optimizing Liposomes for Delivery of Chemotherapeutic Agents to Solid Tumors. Pharmacological Reviews 51, 691-743, 1999 and the references cited therein.
16. Dymock W: Pharmacographia Indica, 2, 398, (1890).
17. Font-Quer P: (1979) Plantas medicinales. Barcelona, Ed. Labor SA.
18. Gabizon A. and Papahadjopoulos D: Liposome formulations with prolonged circulation time in blood and enhanced uptake by tumors. Proc. Natl. Acad. Sci. (Wash.) 85, 6949-53 (1988).
19. Gordon K B, Tajuddin A, Guitart J, Kuzel, T M, Eramo L R, and Von Roenn J: Cancer, 75, 2169-73 (1995).
20. Hanada R, Abe F and Yamauchi T: Phytochemistry, 31, 2459 (1992).
21. Hanada R, Abe F and Yamauchi T: Phytochemistry, 31, 3183 (1992).
22. Hardin J W and Arena J M: (1974) Human poisoning from native a cultivated plants, 2nd ed. Kingsport, Tennessee, Duke University Press.
23. Huxley A: ed-in-chief (1992) The New Horticultural Society dictionary of gardening, vol. 3. London, MacMillan.
24. Julliano R L and Stamp D: Pharmacokinetics of liposome-encapsulated anti-tumor drugs. Biochem. Pharm. 27, 21-27 (1978).
25. Kingsbury J M: (1964) Poisonous plants of the United States and Canada. Englewood Cliffs, N.J. Prentice Hall.
26. Kirtikar K R and Basu B D: Indian Medicinal Plants, Internatiional Book Ditributors, India (1987).
27. Krasso et al: Helv. Chim. Acta, 46, 1691 (1963).
28. Lasic D D, & Martin F: eds. (1995) Stealth Liposomes, CRC, Boca Raton, Fla.
29. Lasic D D and Paphadjopoulos D: Medical Applications of Liposomes, Amsterdam, Elsevier, 1998.
30. Lasic D D: Liposomes: From Physics to Applications. Amsterdam, Elsvier, 1993.
31. Lee et al: “Serum Albumin Beads: An Injectable, Biodegradable System for the Sustained Release of Drugs” Science, 213:233-235 (1981).
32. Leucuta et al: “Albumin microspheres as a drug delivery system for epirubicin: pharmaceutical, pharmacokinetic and biological aspects” International Journal of Pharmaceutics, 41:213-217 (1988).
33. Madden T D, Harrigan P R, Tai L C L, Bally M B, Mayer L D, Redelmeier T E, Loughrey H C, Tilcock C P S, Reinish L W and Cullis P R: The accumulation of drugs within large unilamellar vesicles exhibiting a proton gradient: a survey, Chem. Phys. Lipids, 53, 37-46 (1990).
34. Manna S K, Sah N K, Newman R A, Cisneros A, Aggarwal B B: Cancer Res, 60(14): 3838-47, 2000
35. McConkey D J, Lin Y, Nutt L K, Ozel H Z, Newman R A: Cancer Res, 60(14): 3807-12, 2000
36. Olson F, Hunt C, Szoka F C: Preparation of liposomes of defined size distribution by extrusion through polycarbonate membrane. BioChim. Biophys. Acta, 557, 9-23 (1979).
37. Olson F, Mayhew E, Maslow, Rustum Y and Szoka F: Characterization, toxicity and therapeutic efficacy of adriamycin entrapped in liposomes. Europ. J. Cancer. Clin. Oncol., 18, 167-76 (1982).
38. Pathak S, Multani A S, Narayan S, Kumar V, Newman R A: Anticancer Drugs, 11(6): 455-63, 2000
39. Pearn J: (1987) Oleander poisoning. In: Covacevich J, Davie P and Pearn J: eds (1987) Toxic plants & animals; a guide for Australia. Brisbane, Queensland Museum, pp 37-49.
40. Rahman A., Kessler A., More N, Skies B, Rowden G, Wooley P and Schein P S: Liposomal protection of adriamycin induced cardiotoxicity in mice. Cancer Res., 40, 1532-37 (1982).
41. Reynolds J E F: ed (1989) Martindale: the extra pharmacopoeia, 29th ed. London, The Pharmaceutical Press.
42. Schvartsman S: (1979) Plantas venenosas. Sarvier Sao Paulo.
43. Sharma A, Sharma U S, and Straubinger R M: Paclitaxel-liposomes for intracavitary therapy of intraperitoneal P388 leukemia., Cancer Lett 107: 265-272 (1996).
44. Sharma A, Mayhew E, Bolcsak L, Cavanaugh C and Harmon P: Activity of paclitaxel liposome formulations against human ovarian tumor xenografts. Int J Cancer 71: 103-107 (1997).
45. Shaw D and Pearn J: (1979) Oleander poisoning. Med J Aust, 2: 267-269.
46. Siddiqui B S, Begum S, Hafeez F and Siddiqui S: Phytochemistry, 28, 1187 (1995).
47. Siddiqui B S, Begum S, Siddiqui S and Lichter W: Phytochemistry, 39, 171 (1995).
48. Siddiqui S, Begum S, Hafeez F and Siddiqui B S: Phytochemistry, 28, 1187 (1989).
49. Siddiqui S, Begum S, Siddiqui B S and Hafeez F: J. Nat. Products, 52, 229 (1988).
50. Siddiqui S, Hafeez F, Begum S and Siddiqui B S: J. Nat. Products, 49, 1086 (1987).
51. Siddiqui S, Hafeez F, Begum S and Siddiqui B S: J. Nat. Products, 51, 229 (1988).
52. Siddiqui S, Hafeez F, Begum S, and Siddiqui B S: Phytochemistry, 26, 237 (1987).
53. Siddiqui S, Siddiqui B S, Begum S, and Hafeez F: Plant Med., 55, 292 (1989).
54. Siddiqui S, Siddiqui B S, Hafeez F and Begum S: Planta Med., 53, 424 (1987).
55. Siddiqui S, Siddiqui B S, Hafeez F. and Begun S: Planta Med., 54, 232 (1988).
56. Smith J A, Madden T, Vijjeswarapu M, Newman R A: Biochem Pharmacol, 62(4): 469-72, 2001
57. Smith J A, Madden T, Vijjeswarapu M, Newman R A: Biochem Pharmacol, 62(4): 469-72, 2001
58. Suffness, M: (Ed.) Taxol, Science and Applications, Chapter 9, CRC (1995).
59. Taylor A., McKenna G F and Burlage H M: Texas Reports on Biology and Medicine, 14, 538 (1956).
60. Wang X, Plomley J B, Newman R A, Cisneros A: Anal Chem, 72(15): 3547-52, 2000
61. William S S, Alosco T R, Mayhew E, Lasic D D, Martin F J and Bankert R B: Arrest of human lung tumor xenograft growth in severe combined immunodeficient mice using doxorubicin encapsulated in sterically stabilized liposomes. Cancer Res., 53, 3964-67 (1993).
62. Woodle M C, Allen T M, Mayhew E, and Uster P S: In vivo efficacy of vincristine entrapped in long-circulating liposomes, Proc. Am. Assoc. Cancer Res., 33, 446 (1992).
63. Yeh J Y, Huang W J, Kan S F, Wang P S., Inhibitory effects of digitalis on the proliferation of androgen dependent and independent prostate cancer cells, J. Urol., 2001 166(5): 1937-42.
64. D. Stewart, A. Mobasheri and M. B. A. Djamgoz, Immuno-characterisation of Na⁺,K⁺-ATPase subunit(s) in rat prostate cancer cell lines, The Journal of Physiology (2000), 525P, pp. 96P.
65. Grimes J A, Fraser S P, Stephens G J, Downing J E, Laniado M E, Foster C S, Abel P D, Djamgoz M B., Differential expression of voltage-activated Na⁺ currents in two prostatic tumour cell lines: contribution to invasiveness in vitro., FEBS Lett 1995; 369:290-4.
66. Repke K R H, Schönfeld W, Weiland J, Megges R, Hache, A: In Design of Enzyme Inhibitors as Drugs; Sandler, M.; Smith, H. J., Eds.; Oxford University Press: Oxford, 1989; pp 435-502.
67. Thomas R E, Gray P, Andrews J: In Advances in Drug Research; Testa, L. B., Ed.; Academic Press: London, 1990; Vol. 19, pp 313-575.
68. Blaustein M P: Rev. Physiol. Biochem. Pharmacol. 1974, 70, 33-82.
69. Skou J C: Biochim. Biophys. Acta 1957, 23, 394-401.
70. Albers R W: Annu. Rev. Biochem. 1967, 36, 727-756.
71. Post R L, Kume S, Tobin T, Orcutt T, Sen A K: J. Gen. Physiol. 1969, 54, 306S-326S.
72. Geering K: Curr. Op. Nephrol. Hypertens. 1997, 6, 434-439.
73. Thomas R E: In Molecular Structure and Biological Activity of Steroids; Bohl M, Daux W L, Eds.; CRC Press: Boca Raton, 1992; pp 399-464.
74. Lee C O: Am. J. Physiol. 1985, 249, 367-378.
75. Gillis R A, Quest J A: In Cardiac Glycosides; Erdmann E, Greeff K, Skou J C, Eds.; Steinkopff: Darmstadt, 1986; pp 347-356.
76. Concepción P. Melero, Manuel M and Arturo S F: A Short Review on Cardiotonic Steroids and Their Aminoguanidine Analogues. Molecules 2000, 5, 51-81
77. Wald H, Scherzer P, Popovtzer M M: Enhanced renal tubular ouabain-sensitive ATPase in streptozotocin diabetes mellitus, Am J Physiol 1986; 251:F164-70
78. Jennings P E, Wilkins M R, West M J, Kendall M J, Barnett A H: Sodium transport across erythrocyte membranes in diabetes mellitus. Sodium transport across erythrocyte membranes in diabetes mellitus, Diabetes Res 1986; 3:407-10
79. Kjeldsen K, Braendgaard H, Sidenius P, Larsen J S, Norgaard A: Diabetes decreases Na⁺-K⁺ pump concentration in skeletal muscles, heart ventricular muscle, and peripheral nerves of rat, Diabetes 1987; 36:842-8
80. Baldini P, Incerpi S, Lambert-Gardini S, Spinedi A, Luly P: Membrane lipid alterations and Na⁺-pumping activity in erythrocytes from IDDM and NIDDM subjects, Diabetes 1989; 38:825-31
81. Kuriyama S, Tokudome G, Tomonari H, Utsunomiya Y, Matsui K, Hashimoto T, Sakai O: Differential regulation of cation transport of vascular smooth muscle cells in a high glucose concentration milieu, Diabetes Res Clin Pract 1994; 24:77-84
82. Raccah D, Dadoun F, Coste T, Vague P: Decreased Na/K ATPase ouabain binding sites in red blood cells of patients with insulin-dependent diabetes and healthy north African control subjects: relationship with diabetic neuropathy, Horm Metab Res 1996; 28:128-32
83. Zolese G, Rabini R A, Fumelli P, Staffolani R, Curatola A, Kvasnicka P, Kotyk A, Cester N, Mazzanti L: Modifications induced by insulin-dependent diabetes mellitus on human placental Na⁺/K⁺-adenosine triphosphatase, J Lab Clin Med. 1997; 130:352-3.
84. Gerbi A, Maixent J M, Barbey O, Jamme I, Pierlovisi M, Coste T, Pieroni G, Nouvelot A, Vague P, Raccah D: Alterations of Na,K-ATPase isoenzymes in the rat diabetic neuropathy: protective effect of dietary supplementation with n-3 fatty acids, J Neurochem 1998;71:732-40
85. Wild G E, Thompson J A, Searles L, Turner R, Hasan J, Thomson A B: Small intestinal Na⁺, K⁺-adenosine triphosphatase activity and gene expression in experimental diabetes mellitus, Dig Dis Sci 1999;44:407-14
86. Takigawa T, Yasuda H, Terada M, Maeda K, Haneda M, Kashiwagi A, Kitasato H, Kikkawa R: Increases in K⁺ conductance and Ca²⁺ influx under high glucose with suppressed Na⁺/K⁺-pump activity in rat myelinated nerve fibers, Neuroreport 2000; 11:2547-51
87. Sennoune S, Gerbi A, Duran M J, Grillasca J P, Compe E, Pierre S, Planells R, Bourdeaux M, Vague P, Pieroni G, Maixent J M: Effect of streptozotocin-induced diabetes on rat liver Na⁺/K⁺-ATPase, Eur J Biochem 2000; 267:2071-8.
88. Shiratori O: Growth inhibitory effect of cardiac glycosides and agly cones on neoplastic cells: in vitro and in vivo studies. Gann 1967, 58:521-528
89. Repke K R H: The role of the Na⁺/K⁺ pump in normal and cancer cell proliferation. In: Biomemebranes, Basic and Medical Research (Edited by Benga G, Tager J M) Berlin, Springer Verlag 1988, 161-176
90. Repke K R, Schon R, Megges R, Wetland J, Nissen E, Matthes E: Potential suitability of Na⁺/K⁺ transporting ATPase in pre-screens for anti-cancer agents. Anticancer Drug Des 1995, 10:177-87
91. Cassady J M, Suffness M M: Terpenoid antitumor targets. In: Anti cancer agents based on natural product models (Edited by Cassady J M, Douros J D) New York, Academic Press 1980, 201-269
92. Haux J: Digitoxin is a potential anticancer agent for several types of cancer. Med Hypotheses 1999, 53:543-548
93. Haux J, Lam M, Marthinsen A B L, Strickert T, Lundgren S: Digitoxin, in non toxic concentrations, induces apoptotic cell death in Jurkat T cells in vitro. Z Onkol 1999, 31:14-20
94. Haux J, Solheim O, Isaksen T, Angelsen A: Digitoxin, in non-toxic concentrations, inhibits proliferation and induces cell death in prostate cancer cell lines. Z Onkol 2000, 32:11-16
95. Kawazoe N, Aiuchi T, Masuda Y, Nakajo S, Nakaya K: Induction of apoptosis by bufalin in human tumor cells is associated with a change of intracellular concentration of Na⁺ ions. J Biochem (Tokyo) 1999, 126:278-286
96. Johansson S, Lindholm P, Gullbo J, Larsson R, Bohlin L, Claeson P: Cytotoxicity of digitoxin and related cardiac glycosides in human tumor cells. Anticancer Drugs 2001, 12:475-483
97. Raloff J: Jamming Prostate Cancer's Transmission. Science News 1995, 147, 134.
98. Pienta et al.: Inhibition of Spontaneous Rat Prostate Cancer By Oral Administration of Modified Citrus Pectin. J. Nat'l Cancer Institute, 1995, 87, 348-53.
99. Inohara et al.: Effects of Natural Complex Carbohydrate (Citrus Pectin) On Murine Melanoma Cell Properties Related to Galectin-3 Functions. Glycocon J, 1994, 11, 527-532.
100. Platt, D, and Raz, A: Modulation of the lung colonization of B16-F1 melanoma cells by citrus pectin. J. Natl Cancer Inst, 1992, 84, 438-42.
101. Masaru I, Tsutomu W, and Tadataka N: Bufadienolides and a New Lignan from the Bulbs of Urginea maritima. Chem. Pharm. Bull. 2001, 49, 282-286.
102. Kopp B, Krenn L, Draxler M, Hoyer A, Terkola R, Vallaster P, Robien W: Phytochemistry, 1996, 42, 513-522.
103. Mitsuhashi H, Tanaka O, Nozoe S, Nagai M. (ed.): Chemistry of Organic Natural Products. 4th ed, Nankoudou Press, Tokyo, 1994, 168-174.
104. Schoenfeld W, Weiland J, Lindig C, Masnyk M, Kabat M M, Kurek A, Wicha J, Repke K R H: Naunyn Schmiedeberg's Arch. Pharmacol., 1985, 329, 414-426.
105. Majinda R R T, Waigh R D, Waterman P G: Planta Med., 1997, 63, 188-190.
106. Kopp B, Unterluggauer M, Robien W, Kubelka W: Planta Med., 1990, 56, 193-197.
107. Krenn L, Kopp B, Deim A, Robien W, Kubelka W, Planta Med., 1994, 60, 63-69.
108. Krishna Rao R V, Rangaswami S: Tetrahedron Lett., 1967, 4563-4565.
109. Krenn L, Jambrits M, Kopp B: Planta Med., 1988, 54, 227-232.
110. Tanase T, Nagatsu A, Murakami N, Nagai S, Ueda T, Sakakibara J, Ando H, Hotta Y, Takeya., Asano M: Chem. Pharm. Bull., 1994, 42, 2256-2262.
111. Verbiscar A J, Patel J, Banigan T F, Schatz R A J: Agric. Food Chem., 1986, 34, 973-979.
112. Shimada K, Umezawa E, Nambara T, Kupchan S M: Chem. Pharm. Bull., 1979, 27, 3111-3114.
113. Jha S, Sen S: Phytochemistry, 1981, 20, 524-526.
114. Lichti H, Niklaus P, von Wartburg A: Helv. Chim. Acta, 1973, 56, 2083-2087.
115. Abraham E (2003) Nuclear factor-kB and its role in sepsis-associated organ failure. Journal of Infectious Diseases 187: S364-S369
116. Aradhya S and Nelson D L (2001) NF-kB signaling and human disease. Current Opinions in Genetics and Development 11: 300-306.
117. Arima N and Tei C (2001) HTLV-I Tax related dysfunction of cell cycle regulators and oncogenesis of adult T cell leukemia. Leukemia and Lymphoma 40: 267-278
118. Baghdiguian S, Martin M, Richard I, Pons F, Astier C, Bourg N, Hay R T, Chemaly R, Halaby G, Loiselet J, Anderson L V, Lopez de Munain A, Fardeau M, Mangeat P, Beckmann J S and Lefranc G (1999) Calpain 3 deficiency is associated with myonuclear apoptosis and profound perturbation of the IkB alpha/NF-kB pathway in limb-girdle muscular dystrophy type 2A [published erratum appears in Nat Med July 1999; 5(7):849]. Nature Medicine 5: 503-511
119. Baldwin AS Jr (2001) Series introduction: the transcription factor NF-kB and human disease. Journal of Clinical Investigation 107: 3-6
120. Bargou R C, Leng C, Krappmann D, Emmerich F, Mapara M Y, Bommert K, Royer H-D, Scheidereit C and Dörken (1996) High-level nuclear NF-kB and Oct-2 is a common feature of cultured Hodgkin/Reed-Sternberg cells. Blood 87: 4340-4347
121. Bargou R C, Emmerich F, Krappmann D, Bommert K, Mapara M Y, Arnold W, Royer H D, Grinstein E, Greiner A, Scheidereit C and Dörkin B (1997) Constitutive NF-kB-RelA activation is required for proliferation and survival of Hodgkin's disease tumor cells. Journal of Clinical Investigation 100: 2961-2969
122. Barnes P J (2002) New treatments for COPD. Nature Reviews Drug Discovery 1: 437-446
123. Baumann B, Weber C K, Troppmair J, Whiteside S, Israel A, Rapp U R and Wirth T (2000) Raf induces NF-kB by membrane shuttle kinase MEKK1, a signaling pathway critical for transformation. Proceedings of the National Academy of Sciences USA 97: 4615-4620
124. Bell S, Degitz K, Quirling M, Jilg N, Page S and Brand K (2003) Involvement of NF-kB signalling in skin physiology and disease. Cell Signal 15: 1-7
125. Berenson J R, Ma H M and Vescio R (2001) The role of nuclear factor-kB in the biology and treatment of multiple myeloma. Seminars in Oncology 28: 626-633
126. Besancon F, Atfi A, Gespach C, Cayre Y E and Bourgeade M F (1998) Evidence for a role of NF-kB in the survival of hematopoietic cells mediated by interleukin-3 and the oncogenic TEL/platelet-derived growth factor beta fusion protein. Proceedings of the National Academy of Sciences USA 95: 8081-8086
127. Bian X, Opipari Jr A W, Ratanaproeksa A B, Boltano A E, Lucas P C and Castle V P (2002) Constitutively active NF-kB is required for the survival of S-type neuroblastoma. Journal of Biological Chemistry 277: 42144-42150
128. Bielory L, Kempuraj D and Theoharides T (2003) Topical immunopharmacology of ocular allergies. Current Opinion in Allergy and Clinical Immunology 2: 435-445
129. Brummelkamp T R, Nijman S M B, Cirac A M G and Bernards R (2003) Loss of the cylindromatosis tumour suppressor inhibits apoptosis by activating NF-kB. Nature 424 797-801
130. Cechetto D F (2001) Role of nuclear factor kappaB in neuropathological mechanisms. Progress in Brain Research 132: 391-404
131. Chen F and Shi X (2002) NF-kB, a pivotal transcription factor in silica-induced diseases. Mol Cell Biochem 234-235: 169-176
132. Christman J W, Sadikot R T and Blackwell T S (2000) The role of nuclear factor-kB in pulmonary diseases. Chest 117 1482-1487
133. Chung H Y, Kim H J, Kim K W, Choi J S and Yu B P (2002) Molecular inflammation hypothesis of aging based on the anti-aging mechanism of calorie restriction. Microsc Res Tech 59: 264-272
134. Courtois G and Israel A (2000) NF-kB defects in humans: the NEMO/incontinentia pigmenti connection. Sci STKE November 14 (58): PE1
135. Davis R E, Brown K D, Siebenlist U and Staudt L M (2001) Constitutive nuclear factor kB activity is required for survival of activated B cell-like diffuse large B cell lymphoma cells. Journal of Experimental Medicine 194: 1861-1874
136. Dejardin E, Deregowski V, Chapelier M, Jacobs N, Gielen J, Merville M-P and Bours (1999) Regulation of NF-kB activity by IkB-related proteins in adenocarcinoma cells. Oncogene 18: 2567-2577
137. Dijkstra G, Moshage H and Jansen P L (2002) Blockade of NF-kB activation and donation of nitric oxide: new treatment options in inflammatory bowel disease? Scandinavian Journal of Gastroenterology Suppl 236: 37-41
138. Finco T S, Westwick J K, Norris J L, Beg A A, Der C J and Baldwin Jr A S (1997) Oncogenic Ha-Ras-induced signaling activates NF-kB transcriptional activity, which is required for cellular transformation. Journal of Biological Chemistry 272: 24113-24116
139. Furman R R, Asgary Z, Mascarenhas J O, Liou H C and Schattner E J (2000) Modulation of NF-kB activity and apoptosis in chronic lymphocytic leukemia B cells. Journal of Immunology 164: 2200-2206
140. Gilmore T D, Gapuzan M-E, Kalaitzidis D and Starczynowski D (2002) Rel/NF-kB/IkB signal transduction in the generation and treatment of human cancer. Cancer Letters 181: 1-9
141. Guijarro C and Egido J (2001) Transcription factor-kappaB (NF-kB) and renal disease. Kidney International 59: 415-424
142. Guzman M L, Neering S J, Upchurch D, Grimes B, Howard D S, Rizzieri D A, Luger S M, Jordan C T (2001) Nuclear factor-kB is constitutively activated in primitive human acute myelogenous leukemia cells. Blood 98: 2301-2307
143. Hayashi T and Faustman D (2002) Defective function of the proteasome in autoimmunity: involvement of impaired NF-kB activation. Diabetes Technol Ther 2: 415-428
144. Hayashi S, Yamamoto M, Ueno Y, Ikeda K, Ohshima K, Soma G and Fukushima T (2001) Expression of nuclear factor-kB, tumor necrosis factor receptor type 1, and c-Myc in human astrocytomas. Neurologia Medico-Chirufica 41: 187-195
145. Hiscott J, Kwon H and Genin P (2001) Hostile takeovers: viral appropriation of the NF-kB pathway. Journal of Clinical Investigation 107: 143-151
146. Ho E and Bray T M (1999) Antioxidants, NFkB activation, and diabetogenesis. Proc Soc Exp Biol Med 222: 205-213
147. Holmes-McNary M (2002) Nuclear factor kappaB signaling in catabolic disorders. Curr Opin Clin Nutr Metab Care 5: 255-263
148. Huang S, Pettaway C A, Uehara H, Bucana C D and Fidler I J (2001) Blockade of NF-kB activity in human prostate cancer cells is associated with suppression of angiogenesis, invasion, and metastasis. Oncogene 20: 4188-4197
149. Huang S, Robinson J B, DeGuzman A, Bucanna C D and Fidler I J (2000) Blockade of nuclear factor-kB signaling inhibits angiogenesis and tumorigenicity of ovarian cancer cells by suppressing expression of vascular endothelial growth factor and Interleukin 8. Cancer Research 60: 5334-5339
150. Lavie L (2003) Obstructive sleep apnoea syndrome—an oxidative stress disorder. Sleep Medicine Review 7: 35-51
151. Karin M, Cao Y, Greten F R and Li Z W (2002) NF-kB in cancer: from innocent bystander to major culprit. Nature Reviews Cancer 2: 301-310
152. Kammer G M and Tsokos G C (2002) Abnormal T lymphocyte signal transduction in systemic lupus erythematosus. Current Directions in Autoimmunity 5: 131-150
153. Knecht H, Berger C, Rothenberger S, Odermatt B F and Brousset P (2001) The role of Epstein-Barr virus in neoplastic transformation. Oncology 60: 289-302
154. Kordes U, Krappmann D, Heissmeyer V, Ludwig W D and Scheidereit C (2000) Transcription factor NF-kB is constitutively active in acute lymphoblastic leukemia cells. Leukemia 14: 399-402
155. Kovalenko A, Chabie-Bessia C, Cantarella G, Israel A, Wallach D and Courtois G (2003) The tumour suppressor CYLD negatively regulates NF-kB signalling by deubiquitination. Nature 424: 801-805
156. Lind D S, Hochwald S N, Malaty J, Rekkas S, Hebig P, Mishra G, Moldawer L L, Copeland E M 3^{rd} and Mackay S (2001) Nuclear factor-kB is upregulated in colorectal cancer. Surgery 130: 363-369
157. Madjdpour L, Kneller S, Booy C, Pasch T, Schimmer R C and Beck-Schimmer B (2003) Acid-induced lung injury: role of Nuclear Factor-kB. Anesthesiology 99: 1323-1332
158. Martinez N, Camacho F I, Algara P, Rodriguez A, Dopazo A, Ruiz-Ballesteros E, Martin P, Martinez-Climent J A, Garcia-Conde J, Menarguez J, Solano F, Mollejo M and Piris M A (2003) The molecular signature of mantle cell lymphoma reveals multiple signals favoring cell survival. Cancer Research 63: 8226-8232
159. Mattson M P and Camandola S (2001) NF-kB in neuronal plasticity and neurodegenerative disorders. Journal of Clinical Investigation 107: 247-254
160. Nair A, Venkatraman M, Maliekal T T, Nair B, Karunagaran D (2003) NF-kB is constitutively activated in high-grade squamous intraepithelial lesions and squamous cell carcinomas of the human uterine cervix. Oncogene 22: 50-58
161. Nakshatri H, Bhat-Nakshatri P, Martin D A, Goulet Jr R J and Sledge Jr G W (1997) Constitutive activation of NF-kB during progression of breast cancer to hormone-independent growth. Molecular and Cellular Biology 17: 3629-3639
162. Nakayama H, Ikebe T, Beppu M and Shirasuma K (2001) High expression levels of nuclear factor kB, IkB kinase b and Akt kinase in squamous cell carcinoma of the oral cavity. Cancer 92: 3037-3044
163. Neurath M F, Becker C and Barbulescu K (1998) Role of NF-kB in immune and inflammatory responses in the gut. Gut 43: 856-860
164. Nichols T C, Fischer T H, Deliargyris E N and Baldwin Jr A S (2001) Role of nuclear factor-kappaB (NF-kB) in inflammation, periodontitis, and atherogenesis. Annals of Periodontology 6: 20-29
165. Ondrey F G, Dong G, Sunwoo J, Chen Z, Wolf J S, Crowl-Bancroft C V, Mukaida N and Van Waes C (1999) Constitutive activation of transcription factors NF-kB, AP-1, and NF-IL6 in human head and neck squamous cell carcinoma cell lines that express pro-inflammatory and pro-angiogenic cytokines. Molecular Carcinogenesis 26: 119-129
166. Oya M, Ohtsubo M, Takayanagi A, Tachibana M, Shimizu N and Murai, M (2001) Constitutive activation of NF-kB prevents TRAIL-induced apoptosis in renal cancer cells. Oncogene 20: 3888-3896
167. Pahl A and Szelenyi I (2002) Asthma therapy in the new millennium. Inflammation Research 51: 273-282
168. Palayoor S T, Yourmell M Y, Calderwood S K, Coleman C N and Price B D (1999) Constitutive activation of IkB kinase b and NF-kB in prostate cancer cells is inhibited by ibuprofen. Oncogene 18: 7389-7394
169. Pena A S and Penate M (2002) Genetic susceptibility and regulation of inflammation in Crohn's disease. Relationship with the innate immune system. [Article in English, Spanish] Rev Esp Enferm Dig 94: 351-360
170. Pennington C, Dunn J, Li C, Ha T and Browder W (2000) Nuclear factor kB activation in acute appendicitis: a molecular marker for extent of disease? American Surgery 66: 914-918
171. Purcell N H and Molkentin J D (2003) Is nuclear factor kappaB an attractive therapeutic target for treating cardiac hypertrophy? Circulation 108 638-640
172. Reuter U, Chiarugi A, Bolay H and Moskowitz M A (2003) Nuclear factor-kB as a molecular target for migraine therapy. Headache 43: 426-427
173. Reuther J Y, Reuther G W, Cortez D, Pendergast A M and Baldwin Jr A S (1998) A requirement for NF-kB activation in Bcr-Abl mediated transformation. Genes & Development 12: 968-981
174. Ritchie M E (1998) Nuclear factor kappaB is markedly and selectively activated in humans with unstable angina pectoris. Circulation 98 1707-1713
175. Roshak A K, Callahan J F and Blake S M (2002) Small-molecule inhibitors of NF-kB for the treatment of inflammatory joint disease. Current Opinion in Pharmacology 2: 316-321
176. Roshak A K, Callahan J F, Blake S M, Ross J S, Stagliano N E, Donovan M J, Breitbart R E and Ginsburg G S (2001) Atherosclerosis: a cancer of the blood vessels? American Journal of Clinical Pathology 116: S97-S107
177. Russell J P, Shinohara S, Melillo R M, Castellone M D, Santoro M and Rothstein J L (2003) Tyrosine kinase oncoprotein, RET/PTC3, induces the secretion of myeloid growth and chemotactic factors. Oncogene 22: 4569-4577
178. Santos S C, Monni R, Bouchaert I, Bernard O, Gisselbrecht S, Gouilleux F, Penard-Lacronique V (2001) Involvement of the NF-kB pathway in the transforming properties of the TEL-Jak2 leukemogenic fusion protein. FEBS Letters 497: 148-152
179. Sasaki N, Morisaki T, Hashizume K, Yao T, Tsuneyoshi M, Noshiro H, Nakamura K, Yamanaka T, Uchiyama A, Tanaka M and Katano M (2001) Nuclear factor-kB p65 (RelA) transcription factor is constitutively activated in human gastric carcimoma tissue. Clinical Cancer Research 7: 4136-4142
180. Sclabas G M, Fujioka S, Schmidt C, Evans D B and Chiao P J (2003) NF-kB in pancreatic cancer. International Journal of Gastrointestinal Cancer 33 15-26
181. Seppanen M and Vihko K K (2000) Activation of transcription factor NF-kB by growth inhibitory cytokines in vulvar carcinoma cells. Immunology Letters 74: 103-109
182. Shaffer A L, Rosenwald A and Staudt L M (2002) Lymphoid malignancies: the dark side of B-cell differentiation. Nature Review Immunology 2: 920-933
183. Sovak M A, Bellas R E, Kim D W, Zanieski G J, Rogers A E, Traish A M and Sonenshein G E (1997) Aberrant nuclear factor-kB/Rel expression and the pathogenesis of breast cancer. Journal of Clinical Investigation 100: 2952-2960
184. Staudt L M (2000) The molecular and cellular origins of Hodgkin's disease. Journal of Experimental Medicine 191: 207-212
185. Sutter A P, Zeitz M and Scherubl H (2004) Recent results in understanding molecular pathways in the medical treatment of esophageal and gastric cancer. Onkologie 27 17-21
186. Tai D I, Tsai S L, Chang Y H, Huang S N, Chen T C, Chang K S and Liaw Y F (2000) Constitutive activation of nuclear factor kB in hepatocellular carcinoma. Cancer 89: 2274-2281
187. Toledo-Pereyra L and Lopez-Neblina F (2002) New advances in the molecular biology of ischemia/reperfusion: MAPK pathways. Tissue Antigens 60: 552
188. Trompouki E, Hatzivassillou E, Tsichritzis T, Farmer H, Ashworth A and Mosialos G (2003) CYLD is a deubiquitinating enzyme that negatively regulates NF-kB activation by TNFR family members. Nature 424 793-796
189. Valen G, Yan Z Q and Hansson G K (2001) Nuclear factor kB and the heart. Journal of the American College of Cardiology 38: 307-314
190. Visconti R, Cerutti J, Battista S, Fedele M, Trapasso F, Zeki K, Miano M P, de Nigris F, Casalino L, Curcio F, Santoro M and Fusco A (1997) Expression of the neoplastic phenotype by human thyroid carcinoma cell lines requires NFkB p65 protein expression. Oncogene 15: 987-1994
191. Wang W, Abbruzzese J L, Evans D B, Larry L, Cleary K and Chiao P J (1999) The NF-kB RelA transcription factor is constiutively activated in human pancreatic adenocarcinoma cells. Clinical Cancer Research 5: 119-127
192. Weber C K and Adler G (2001) From acinar cell damage to systemic inflammatory response: current concepts in pancreatitis. Pancreatology 1: 356-362
193. Whitehead I P, Lambert Q T, Glaven J A, Abe K, Rossman K L, Mahon G M, Trzaskos J M, Kay R, Campbell S L and Der C J (1999) Dependence of Dbl and Dbs transformation on MEK and NF-kB activation. Molecular and Cellular Biology 19: 7759-7770
194. Wilson S H, Caplice N M, Simari R D, Holmes D R, Jr., Carlson P J and Lerman A (2000) Activated nuclear factor-kB is present in the coronary vasculature in experimental hypercholesterolemia. Atherosclerosis 148: 23-30
195. Wratten M L, Brendolan A, Ronco C, La Greca G and Tetta C (2001) Should we target signal pathways instead of single mediators in the treatment of sepsis? Contrib Nephrol 132: 400-414
196. Yamamoto Y and Gaynor R B (2001) Role of the NF-kB pathway in the pathogenesis of human disease states. Curr Mol Med 1: 287-296
197. Yang C W, Wu M S and Pan M J (2001) Leptospirosis renal disease. Nephrol Dial Transplant 16 Suppl 5: 73-77
198. Yang J and Richmond A (2001) Constitutive IkB kinase activity correlates with nuclear factor-kB activation in human melanoma cells. Cancer Research 61: 4901-4909
199. Yuan M, Korzstantopoulos N, Lee J, Hansen L, Li Z W, Karin M and Shoelson S E (2001) Reversal of obesity- and diet-induced insulin resistance with salicylates or targeted disruption of IKKb. Science 293: 1673-1677

We claim:

1. A method for the treatment of skin hyperproliferative, inflammatory or infectious disorders in a patient, the method comprising administering to affected skin an effective amount of a composition comprising at least one digitalis glycoside.

2. The method of claim 1, wherein the digitalis glycoside composition further comprises a cyclodextrin.

3. The method of claim 2, wherein the cyclodextrin is amorphous cyclodextrin.

4. The method of claim 1, wherein the composition is comprised in a topical formulation.

5. The method of claim 4, wherein the topical formulation is a cream, lotion, spray, wipe, or drop formulation.

6. The method of claim 1, wherein the composition comprises one or more additional pharmaceutical agents.

7. The method of claim 6, wherein the one or more additional pharaceutical agents is one or more fungicidal or fungistatic agents.

8. The method of claim 6, wherein the one or more additional pharaceutical agents is one or more bacteriocidal or bacteriostatic agents.

9. The method of claim 6, wherein the one or more additional pharaceutical agents is one or more viricidal or viristatic agents.

10. The method of claim 6, wherein the one or more additional pharaceutical agents is one or more cytotoxic agents.

11. The method of claim 1, wherein the composition is a pharmaceutical composition further comprising one or more pharmaceutically acceptable excipients.

12. The method of claim 11, wherein the excipients include one or more pharmaceutically acceptable antioxidants.

13. The method of claim 12, wherein the antioxidant is ascorbic acid, sodium ascorbate, sodium bisulfite, sodium metabisulfate, curcurmin, curcumin derivatives, ursolic acid, resveratrol, resveratrol derivatives, alpha-lipoic acid or monothioglycerol.

14. The method of claim 11, wherein the excipients include one or more pharmaceutically acceptable preservatives and/or buffering agents.

15. The method of claim 14, wherein the buffering agent is monobasic and dibasic sodium phosphate, sodium benzoate, potassium benzoate, sodium citrate, sodium acetate or sodium tartrate.

16. The method of claim 14, wherein the preservative is methylparaben, methylparaben sodium, propylparaben, propylparaben sodium, benzalkonium chloride or benzthonium chloride.

17. The method of claim 1, wherein the composition comprises one or more pharmaceutically acceptable polysaccharides.

18. The method of claim 17, wherein the polysaccharide is dextran sulfate, pectin, modified pectin, insoluble 1,3-β-D glucan, micronized 1,3-β-D glucan, soluble 1,3-β-D glucan, phosphorylated 1,3-β-D glucan, aminated 1,3-β-D glucan and carboxymethylated 1,3-β-D glucan, sulfated 1,3-β-D glucan, insoluble 1,3/1,6-β-D glucan, micronized 1,3/1,6-β-D glucan, soluble 1,3/1,6-β-D glucan, phosphorylated 1,3/1,6-β-D glucan, aminated 1,3/1,6-β-D glucan and carboxymethylated 1,3/1,6-β-D glucan or sulfated 1,3/1,6-β-D glucan.

19. The method of claim 2 wherein the patient is a human.

20. The method of claim 1, wherein the digitalis glycoside is oleandrin, neriifolin, odoroside A and H, ouabain (G-strophantin), cymarin, sarmentocymarin, periplocymarin, K-strophantin, thevetin A, cerberin, peruvoside, thevetosin, thevetin B, tanghinin, deacetyltanghinin, echujin, hongheloside G, honghelin, periplocin, strophantidol, nigrescin, uzarin, calotropin, cheiroside A, cheirotoxin, euonoside, euobioside, euomonoside, lancetoxin A and B, kalanchoside, bryotoxin A-C, bryophyllin B, cotiledoside, tyledoside A-D, F and G, orbicuside A-C, alloglaucotoxin, corotoxin, coroglaucin, glaucorin, scillarene A and B, scilliroside, scilliacinoside, scilliglaucoside, scilliglaucosidin, scillirosidin, scillirubrosidin, scillirubroside, proscillaridin A, rubelin, convalloside, convallatoxin, bovoside A, glucobovoside A, bovoruboside, antiarin A, helleborin, hellebrin, adonidin, adonin, adonitoxin, thesiuside, digitoxin, gitoxin, gitalin, digoxin, F-gitonin, digitonin, lanatoside A-C, bufotalin, bufotalinin, bufotalidin, pseudobufotalin, acetyl-digitoxin, acetyl-oleandrin, beta-methyldigoxin or alpha-methyldigoxin.

21. The method of claim 20, wherein the digitalis glycoside is oleandrin.

22. The method of claim 20, wherein the digitalis glycoside is odoroside A or odoroside H.

23. The method of claim 20, wherein the digitalis glycoside is digitoxin.

24. The method of claim 20, wherein the digitalis glycoside is proscillaridin A.

25. The method of claim 24, wherein the digitalis glycoside is methyl-proscillaridin A.

26. The method of claim 20, wherein the digitalis glycoside is neriifolin.

27. The method of claim 1, wherein the composition comprises from 0.01 mg per gram to 10 mg per gram by weight of the digitalis glycoside.

28. The method of claim 27, wherein the composition comprises from 0.04 mg per gram to 2 mg per gram by weight of the digitalis glycoside.

29. The method of claim 1, wherein the composition is administered orally, nasally, topically, rectally or vaginally.

30. The method of claim 3, wherein the amorphous cyclodextrin has a degree of substitution of 2 to 7.

31. The method of claim 3, wherein the ratio by weight of digitalis glycoside to amorphous cyclodextrin is 0.01 to 1.

32. The method of claim 1, wherein the skin disorder is a hyperproliferative disorder.

33. The method of claim 1, wherein the skin disorder is an inflammatory disorder.

34. The method of claim 33, wherein the inflammatory disorder is acne, psoriasis, dandruff, decubitus and diabetic ulcers, skin lesions of lupus erythematosus, erythema multiforme, folliculitis, and rosacea.

35. The method of claim 1, wherein the skin disorder is an infectious disorder.

36. The method of claim 35, wherein the infectious disorder is cutaneous leishmaniasis, Tinea spp. infections, Candida spp. infections, Coccidioides spp. infections, moniliasis, dermatological Staphylococcus infections, infections of the eye and conjunctiva, Treponema infections including syphillus and yaws, dermatological lesions due to Herpes Simplex virus types I and II, and dermatologic pathologies due to tuberculosis infections.

37. A method of for the systemic treatment of diabetes types I and II, muscular dystrophy, meningitis due to bacterial or fungal pathogens, pulmonary infections, asthma, leptospirosis renal disease, gut diseases, periodontal diseases, lupus erythematosis, systemic leishmaniasis, systemic Coccidioides spp. infections, Crohns disease, inflammatory bowel disease, irritable bowel syndrome, or human immunodeficiency virus infections (AIDS), the method comprising administering to an affected individual an effective amount of a composition comprising at least one digitalis glycoside.

38. The method of claim 37, wherein the digitalis glycoside composition further comprises a cyclodextrin.

39. The method of claim 38, wherein the cyclodextrin is amorphous cyclodextrin.

40. The method of claim 37, wherein the composition is comprised in an oral formulation and the formulation is administered orally.
